SEQUENCE LISTING (77 sequences)

SEQ ID NO: 1 - protein, 677 aa, murine leukemia virus GP70
MDTRRPRQGSDHTPDKTIMESTTLSKPFKNQVNPWGPLIVLLILGGVNPVALGNSPHQVFNLSWEVTNGDRETVWAITGNHPLWTWWPDLTPDLCMLALHGPFSPPPGPPCCSGSSDSTPGCSRDCEEPLTSYTPRCNTAWNRLKLSKVTHAHNEGFYVCPGPHRPRWARSCGGPESFYCASWGCETTGRASWKPSSSWDYITVSNNLTSDQATPVCKGNEWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLCLVSGPPYYEGVAVLGTYSNHTSAPANCSVASQHKLTLSEVTGQGLCIGAVPKTHQVLCNTTQKTSDGSYYLAAPTGTTWACSTGLTPCISTTILDLTTDYCVLVELWPRVTYHSPSYVYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCFYADHTGLVRDSMAKLRERLSQRQKLFESQQGWFEGLFNKSPWFTTLISTIMGPLIILLLILLFGPCILNRLVQFIKDRISVVQALVLTQQYHQLKTIGDCKSRE

SEQ ID NO: 2 - protein, 15 aa, murine leukemia virus GP70
VNPWGPLIVLLILGG

SEQ ID NO: 3 - protein, 9 aa, murine leukemia vius GP70
NPWGPLIVL

SEQ ID NO: 4 - protein, 15 aa, murine leukemia virus GP70
PWGPLIVLLILGGVN

SEQ ID NO: 5 - protein, 9 aa, murine leukemia virus GP70
GPLIVLLIL

SEQ ID NO: 6 - protein, 15 aa, murine leukemia virus GP70
GPLIVLLILGGVNPV

SEQ ID NO: 7 - protein, 15 aa, murine leukemia virus GP70
LGGVNPVALGNSPHQ

SEQ ID NO: 8 - protein, 9 aa, murine leukemia virus GP70
NSPHQVFNL

SEQ ID NO: 9 - protein, 15 aa, murine leukemia virus GP70
NLSWEVTNGDRETVW

SEQ ID NO: 10 - protein, 15 aa, murine leukemia virus GP70
NGDRETVWAITGNHP

SEQ ID NO: 11 - protein, 9 aa, murine leukemia virus GP70
TPDLCMLAL

SEQ ID NO: 12 - protein, 9 aa, murine leukemia virus GP70
SYWGLEYRA

SEQ ID NO: 13 - protein, 9 aa, murine leukemia virus GP70
SYTPRCNTA

SEQ ID NO: 14 - protein, 15 aa, murine leukemia virus GP70
RCNTAWNRLKLSKVT

SEQ ID NO: 15 - protein, 15 aa, murine leukemia virus GP70
NTAWNRLKLSKVTHA

SEQ ID NO: 16 - protein, 15 aa, murine leukemia virus GP70
WNRLKLSKVTHAHNE

SEQ ID NO: 17 - protein, 15 aa, murine leukemia virus GP70
NEGFYVCPGPHRPRW

SEQ ID NO: 18 - protein, 9 aa, murine leukemia virus GP70
RSCGGPESF

SEQ ID NO: 19 - protein, 15 aa, murine leukemia virus GP70
CETTGRASWKPSSSW

SEQ ID NO: 20 - protein, 9 aa, murine leukemia virus GP70
KPSSSWDYI

SEQ ID NO: 21 - protein, 9 aa, murine leukemia virus GP70
DYITVSNNL

SEQ ID NO: 22 - protein, 15 aa, murine leukemia virus GP70
ITVSNNLTSDQATPV

SEQ ID NO: 23 - protein, 15 aa, murine leukemia virus GP
TGHWWGLRLYVSGHD

SEQ ID NO: 24 - protein, 9 aa, murine leukemia virus GP70
LYVSGHDPG

SEQ ID NO: 25 - protein, 9 aa, murine leukemia virus GP70
VPIGPNPVL

SEQ ID NO: 26 - protein, 15 aa, murine leukemia virus GP70
NPVLSDRRPPSRPRP

SEQ ID NO: 27 - protein, 9 aa, murine leukemia virus GP70
NSTPTETPL

SEQ ID NO: 28 - protein, 9 aa, murine leukemia virus GP70
TPTETPLTL

SEQ ID NO: 29 - protein, 9 aa, murine leukemia virus GP70
PPAGVENRL

SEQ ID NO: 30 - protein, 15 aa, murine leukemia virus GP70
KTQECWLCLVSGPPY

SEQ ID NO: 31 - protein, 15 aa, murine leukemia virus GP70
PPYYEGVAVLGTYSN

SEQ ID NO: 32 - protein, 9 aa, murine leukemia virus GP70
PYYEGVAVL

SEQ ID NO: 33 - protein, 15 aa, murine leukemia virus GP70
YSNHTSAPANCSVAS

SEQ ID NO: 34 - protein, 15 aa, murine leukemia virus GP70
NHTSAPANCSVASQH

SEQ ID NO: 35 - protein, 9 aa, murine leukemia virus GP70
CSVASQHKL

SEQ ID NO: 36 - protein, 15 aa, murine leukemia virus GP70
VASQHKLTLSEVTGQ

SEQ ID NO: 37 - protein, 15 aa, murine leukemia virus GP70
SDGSYYLAAPTGTTW

SEQ ID NO: 38 - protein, 15 aa, murine leukemia virus GP70
WACSTGLTPCISTTI

SEQ ID NO: 39 - protein, 9 aa, murine leukemia virus GP70
TPCISTTIL

SEQ ID NO: 40 - protein, 9 aa, murine leukemia virus GP70
SPSYVYHQF

SEQ ID NO: 41 - protein, 15 aa, murine leukemia virus GP70
SPSYVYHQFERRAKY

SEQ ID NO: 42 - protein, 15 aa, murine leukemia virus GP70
YHQFERRAKYKREPV

SEQ ID NO: 43 - protein, 9 aa, murine leukemia virus GP70
KYKREPVSL

SEQ ID NO: 44 - protein, 15 aa, murine leukemia virus GP70
KYKREPVSLTLALLL

SEQ ID NO: 45 - protein, 9 aa, murine leukemia virus GP70
EPVSLTLAL

SEQ ID NO: 46 - protein, 15 aa, murine leukemia virus GP70
EPVSLTLALLLGGLT

SEQ ID NO: 47 - protein, 15 aa, murine leukemia virus GP70
PVSLTLALLLGGLTM

SEQ ID NO: 48 - protein, 15 aa, murine leukemia virus GP70
VSLTLALLLGGLTMG

SEQ ID NO: 49 - protein, 15 aa, murine leukemia virus GP70
GLTMGGIAAGVGTGT

SEQ ID NO: 50 - protein, 15 aa, murine leukemia virus GP70
GTGTTALVATQQFQQ

SEQ ID NO: 51 - protein, 9 aa, murine leukemia virus GP70
TNLEKSLTS

SEQ ID NO: 52 - protein, 15 aa, murine leukemia virus GP70
RRGLDLLFLKEGGLC

SEQ ID NO: 53 - protein, 15 aa, murine leukemia virus GP70
LKEECCLYADHTGLV

SEQ ID NO: 54 - protein, 15 aa, murine leukemia virus GP70
CCLYADHTGLVRDSM

SEQ ID NO: 55 - protein, 9 aa, murine leukemia virus GP70
LYADHTGLV

SEQ ID NO: 56 - protein, 15 aa, murine leukemia virus GP70
ADHTGLVRDSMAKLR

SEQ ID NO: 57 - protein, 15 aa, murine leukemia virus GP70
RDSMAKLRERLSQRQ

SEQ ID NO: 58 - protein, 15 aa, murine leukemia virus GP70
MAKLRERLSQRQKLF

SEQ ID NO: 59 - protein, 15 aa, murine leukemia virus GP70
ERLSQRQKLFESQQG

SEQ ID NO: 60 - protein, 9 aa, murine leukemia virus GP70
WFTTLISTI

SEQ ID NO: 61 - protein, 15 aa, murine leukemia virus GP70
MGPLIILLLILLFGP

SEQ ID NO: 62 - protein, 15 aa, murine leukemia virus GP70
PLIILLLILLFGPCI

SEQ ID NO: 63 - protein, 15 aa, murine leukemia virus GP70
ILLFGPCILNRLVQF

SEQ ID NO: 64 - protein, 14 aa, murine leukemia virus GP70
LVQFIKDRISVVQA

SEQ ID NO: 65 - protein, 15 aa, murine leukemia virus GP70
QFIKDRISVVQALVL

SEQ ID NO: 66 - protein, 15 aa, murine leukemia virus GP70
KDRISVVQALVLTQQ

SEQ ID NO: 67 - protein, 9 aa, murine leukemia virus GP70
ISVVQALVL

SEQ ID NO: 68 - protein, 27 aa, murine leukemia virus GP70
SYTPRCNTAWNRLKLSKVTHAHNEKFA

SEQ ID NO: 69 - protein, 37 aa, murine leukemia virus GP70
FAKETTGRASWKPSSSWDYITVSNNLTSDQATPVKKK

SEQ ID NO: 70 - protein, 40 aa, murine leukemia virus GP70
SPSYVYHQFERRAKYKREPVSLTLALLLGGLTMGKKKKFA

SEQ ID NO: 71 - protein, 29 aa, murine leukemia virus GP70
SPSYVYHQFKKKLVQFIKDRISVVQAKFA

SEQ ID NO: 72 - protein, 9 aa, murine leukemia virus GP70
SPSYVYHQF

SEQ ID NO: 73 - protein, 9 aa, murine leukemia virus GP70
SPSYVYHQF

SEQ ID NO: 74 - protein, 9 aa, artificial peptide
  peptide
RPQASGVYM

SEQ ID NO: 75 - protein, 30 aa, chicken ovalbumin
ISQAVHAAHAEINEAGRESIINFEKLTEWT

SEQ ID NO: 76 - protein, 17 aa, chicken ovalbumin
ISQAVHAAHAEINEAGR

SEQ ID NO: 77 - protein, 8 aa, chicken ovalbumin
SIINFEKL

(19) United States
(12) Patent Application Publication
(10) Pub. No.: US 2021/0236623 A1
(43) Pub. Date: Aug. 5, 2021

(54) T-CELL INDUCING VACCINE COMPOSITION COMBINATION AND USES THEREOF

(71) Applicant: Altimmune, Inc., Gaithersburg, MD (US)

(72) Inventors: Bertrand Georges, Leeds (GB); Scot Roberts, Myersville, MD (US)

(21) Appl. No.: 17/044,952

(22) PCT Filed: Apr. 4, 2019
(86) PCT No.: PCT/US2019/025902

§ 371 Date: Oct. 2, 2020

Related U.S. Application Data
(60) Provisional application No. 62/652,478, filed on Apr. 4, 2018.
(60) Provisional application No. 62/652,484, filed on Apr. 4, 2018.

(51) Int. Cl. A61K 39/21; A61K 39/395; A61K 39/00; A61K 38/16; A61P 35/00; C12N 7/00

(52) U.S. Cl. CPC A61K 39/21; A61K 39/3955; A61K 39/0011; A61K 38/162; A61P 35/00; C12N 2740/13034; A61K 2039/545; A61K 2039/505; A61K 2039/60; A61K 2039/5256; C12N 2710/10043; C12N 7/00

(57) ABSTRACT

Provided herein are vaccine combinations and methods for enhancing an antigen specific T cell induced response in a subject in need thereof. The methods combine systemic vaccination with a first composition containing a non-replicating viral vector encoding an antigen or immunogen containing one or more CD8+ T cell epitopes; and/or a second composition with micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen to induce antigen specific CD8+ T cells; and optionally a third composition comprising an immune modulator composition.

### CROSS-REFERENCE TO RELATED APPLICATIONS

This application claims the benefit of U.S. Provisional Patent Application Nos. 62/652,478, filed on 4 Apr. 2018; and 62/652,484 filed 4 Apr. 2018, the contents of which are each incorporated herein by reference in their entirety.

### FIELD OF THE INVENTION

This application pertains generally to a vaccine combination comprising a prime dose, and/or a heterologous boost dose, and optionally an immune inhibitor dose, and methods for inducing an antigen specific CD8+ T cell response.

### BACKGROUND OF THE INVENTION

Vaccines are limited in their ability to promote robust CD4+ and/or CD8+ T cells responses which are known to play an important role against diseases induced by intracellular pathogens and cancers. The sole exception, the BCG vaccine against tuberculosis, appears to protect primarily by induction of T cells.

Substantial efforts have been made for the generation of T-cell-inducing vaccines designed to induce CD4+ and/or CD8+ T cells of sufficient magnitude and effector function that directly contribute to clearance of infected or tumor cells. Vaccines that use synthetic peptides or DNA combined with a delivery system or recombinant viral vectors are of particular interest for the induction of cell-mediated immunity by offering the ability of delivering or expressing an antigen intracellularly. However, homologous prime-boost regimen, in which a prime dose and boost dose of an antigen/immunogens/peptides are presented to the immune system via the same delivery carriers and/or vectors, have not been able to demonstrate significant clinical efficacy due to limited ability in the generation of robust and durable anti-viral and anti-tumor T cell immunity.

Heterologous prime-boost vaccination, using different delivery carriers and/or vectors, represents a promising strategy compared to homologous prime-boosting for the induction of T cell immunity due to: i) diminished anti-viral vector antibody responses known to interfere with immunity against target antigen through the clearance of vaccine via vaccine-antibody immune complexes; and ii) the potential for different vaccine technologies to stimulate the immune response differently and work synergistically. Heterologous prime-boost approaches have looked at the vaccines of various compositions but have recently mainly focused on DNA/viral vector or viral vector/viral vector combinations for clinical development. With the prospect of stimulating strong and durable T cell immunity, Heterologous prime/boost vaccine strategies are of particular interest for acute and chronic viral infection, cancer, allergy and autoimmunity.

Vaccines against cancer or chronic viral infection represent an attractive approach to provide high specificity, a favorable safety profile, off-the-shelf applicability and the promise of life-long anti-tumor immunity compared to other therapies. Even though T cell vaccines have been greatly improved, overall, they still fail to provide any clinical benefit as monotherapy in patients with advanced cancers or chronic viral infections.

In the context of cancer and chronic viral infection, immunosuppressive mechanisms prevent or reduce the effector activity of antigen-specific T cells resulting from vaccine immunotherapy. These immunosuppressive mechanisms may be exerted, either directly or indirectly, by suppressive myeloid cell, tumor-associated macrophages, T regulatory cells and/or inhibitory receptors expressed on T cells. Consequently, an anti-immunosuppressor, such as checkpoint blockade inhibitors, suppressive myeloid cell inhibitors or compounds involved in the reprogramming or repolarization of suppressive myeloid cells represents a class of therapeutic drugs that have the potential to improve upon the induction and antiviral or antitumoral function of antigen-specific T cells resulting from vaccine immunotherapy. In addition, immunoactivators such as agents targeting co-stimulatory receptors expressed by T cells, cytokines or immune stimulants may also be used to improve upon the induction and antiviral or antitumoral function of antigen-specific T cells resulting from vaccine immunotherapy.

Thus, there is a need for more robust vaccine compositions that induce cell mediate immune responses, particularly in the context of cancer and chronic infections.

### SUMMARY OF THE INVENTION

Herein some embodiments provided include vaccine combinations and methods for administering a heterologous prime boost dosing regimen, wherein the vaccine composition of the prime dose is different than the boost dose provided that each dose comprises one or more CD8+ T cell epitopes that are the same. In other words, the vaccine compositions comprise polypeptides with at least one CD8+ T cell epitope in common. The present vaccine compositions are T cell inducing vaccine compositions; vaccines designed to induce CD4+ and/or CD8+ T cells of sufficient magnitude and necessary phenotype or effector function that directly contribute to pathogen or tumor clearance via cell-mediated effector mechanisms as compared to only CD4+ T cell help for B cells leading to protective antibody responses.

In certain embodiments is provided a vaccine combination comprising a) a first composition comprising a non-replicating viral vector encoding an antigen or immunogen containing one or more CD8+ T cell epitopes; and, b) a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen. See Example 1. Either of the first or second composition may be the prime composition or boost composition. In certain embodiments, the vaccine combination may further comprise an immune modulator comprising an anti-immunosuppressor or an immunoactivator.

In embodiments, the non-replicating viral vector is an adenovirus vector, an alphavirus vector, a herpesvirus vector, a measles virus vector, a poxviruses vector, or a vesicular stomatitis virus vector. In certain embodiments, the non-replicating viral vector is an E1 and E3 deleted adenovirus vector.

In exemplary embodiments, the vaccine combination is used in methods to induce an immune response as a cancer vaccine or chronic infection vaccine (prophylactically or therapeutically). For cancer vaccines, the challenge is not so much to find patient specific antigens (although that is still important) but to develop more immunogenic methods of inducing the required immune response, which requires breaking immunological tolerance to self-antigens. Applicants herein provide a highly immunogenic dosing regimen, wherein the heterologous dosing regimen is synergist as compared to homologous dosing in a murine tumor model tested herein. See Example 3 and FIGS. 1-2.

Accordingly, provided herein are methods for inducing an immune response (e.g., an antigen CD8+ T cell response) in a subject in need thereof, comprising administering a first composition comprising a non-replicating viral vector encoding an antigen or immunogen containing one or more CD8+ T cell epitopes; and, administering a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen. One of the first composition or the second composition is administered as a prime dose and one of the other first composition or the second composition is administered as a boost dose, provided both the first and second compositions are administered.

Herein, further embodiments provided include vaccine combinations and methods for administering those compositions of the vaccine combination, wherein the combination comprises a T cell inducing vaccine composition and a composition comprising an immune inhibitor selected from an immune checkpoint inhibitor or a myeloid-derived suppressor cell (MDSC) inhibitor. T cell inducing vaccine compositions are vaccines designed to induce CD4⁺ and/or CD8⁺ T cells of sufficient magnitude and necessary phenotype or effector function that directly contribute to pathogen or tumor clearance via cell-mediated effector mechanisms as compared to only CD4⁺ T cell help for B cells leading to protective antibody responses. In embodiments, the vaccine composition comprises a polypeptide with at least one CD8+ T cell epitope. In certain embodiments, the present T cell inducing vaccine composition comprises either a non-replicating viral vector or a fluorocarbon linked peptide(s).

In certain embodiments is provided a vaccine combination comprising a first composition comprising a non-replicating viral vector encoding an antigen or immunogen containing one or more CD8+ T cell epitopes; or, b) a second composition comprising micelles containing fluorocarbon-linked peptides, wherein each peptide linked to the fluorocarbon is: i) 15 to 75 amino acid residues long; ii) from an antigen or immunogen; and, iii) contains one or more of the CD8+ T cell epitopes of the antigen or immunogen; and, c) a third composition comprising an immune modulator selected from an anti-immunosuppressor or an immunoactivator.

In embodiments, the anti-immunosuppressor targets PD1, PDL1, PDL2, CD28, CD80, CD86, CTLA4, B7RP1, ICOS, B7RPI, B7-H3, B7-H4, BTLA, HVEM, KIR, TCR, LAG3, CD 137, CD137L, OX40, OX40L, CD27, CD70, CD40, CD40L, TIM3, GAL1, GAL3, GALS, ADORA, CD276, VTCN1, IDO1, KIR3DL1, HAVCR2, VISTA, CD244, ADAM17, COX2, PGE-2, iNOS2, PDE5, c-kit, ARG1, PI3K, CSF-1R, Caspase-8, CCL2, RON, ROS, or S100A8/A9. In embodiments, the anti-immunosuppressor is Pembrolizumab (KEYTRUDA), Nivolumab (OPDIVO), Cemiplimab (LIBTAYO), Atezolizumab (TECENTRIQ), Avelumab (BAVENCIO), Durvalumab (IMFINZI) Ipilimumab (YERVOY), REGN2810, BMS-936558, SHR1210, KN035, IBI308, PDR001, BGB-A317, BCD-100 or JS001.

In certain other embodiments, the anti-immunosuppressor is an MDSC inhibitor targeting PGE-2, COX2, NOS2, ARG1, PI3K, CSF-1R, Caspase-8, CCL2, RON, ROSS100A8/A9 or liver-X nuclear receptor. In embodiments, the MDSC inhibitor is PF-5480090, INCB7839, nitro-aspirine, SC58236, Celecoxib, IPI-549, PLX3397, BLZ945, GW2580, RG7155, IMC-CS4, AMG-820, ARRY-382, sildenafil, tadalafil, vardenafil, N-hydroxy-nor-L-Arg, imatinib, z-IETD-FMK, trabectedin, Emricasan, anti-CCL2 antibody (carlumab or ABN912), Tasquinimod, ASLAN002, IMC-RON8, or GW3965.

In certain embodiments, the immunoactivator targets Toll-like receptor (TLR) 3, TLR4, TLR5, TLR7, TLR8, TLR9, NOD1, NOD2, STING, cGAS, IFR3, 1L-2 receptor, IL12 receptor or IFN-alpha receptor. In certain embodiments, the immunoactivators is IMO-2125, SD-101, DV281, ADZ1419, PF-3512676 (AGATOLIMOD), CMP-001, Lefitolimod, IC31, MEDI9197, RO6864018, RO7020531, GS-9620, AZD8848, LFX453, CV 8102, Moto mod (VTX-2337), BDB001, HILTONOL, KIN131A, MK-4621 (RGT100), Inarigivir (SB9200), MIW815 (ADU-S100), MK-1454, BMS-986301, SB 11285, IL-2, IL-12, or IFN-α.

In embodiments, the non-replicating viral vector is an adenovirus vector, an alphavirus vector, a herpesvirus vector, a measles virus vector, a poxviruses vector, or a vesicular stomatitis virus vector. In certain embodiments, the non-replicating viral vector is an E1 and E3 deleted adenovirus vector.

In certain embodiments provided herein is a vaccine combination comprises a first composition comprising a non-replicating viral vector encoding a peptide of an antigen or immunogen containing one or more CD8+ T cell epitopes; and, a third composition comprising an immune modulator selected from an anti-immunosuppressor or an immunoactivator. In certain other embodiments provided herein is a vaccine composition comprising a second composition comprising micelles containing fluorocarbon-linked peptides, wherein each peptide linked to the fluorocarbon is: i) 15 to 75 amino acid residues long; ii) from an antigen or immunogen; and, iii) contains one or more of the CD8+ T cell epitopes of an antigen or immunogen; and, a third composition comprising an immune modulator selected from an anti-immunosuppressor or an immunoactivator.

In exemplary embodiments, the vaccine combination is used in methods to induce an immune response as a cancer vaccine or chronic infection vaccine (prophylactically or therapeutically). Applicants herein provide a highly immunogenic dosing regimen, wherein the T cell inducing vaccine is synergist with an immune modulator selected from an immune checkpoint inhibitor or a myeloid-derived suppressor cell (MDSC) inhibitor as compared to immune modulator or T cell inducing vaccine composition alone in a murine tumor model tested herein. See Example 5 and 7.

Accordingly, provided herein are methods for inducing an immune response (e.g., an antigen CD8+ T cell response) in a subject in need thereof, comprising administering a composition comprising a non-replicating viral vector encoding a peptide of an antigen or immunogen containing one or more CD8+ T cell epitopes or administering a composition comprising micelles containing fluorocarbon-linked peptides, wherein each peptide linked to the fluorocarbon is: i) 15 to 75 amino acid residues long; ii) from an antigen or immunogen; and, iii) contains one or more of the CD8+ T cell epitopes of an antigen or immunogen; and, administering separately a composition comprising an immune modulator selected from an anti-immunosuppressor or an immunoactivator.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows the cumulative IFN-γ ELISpot responses to the four peptides expressed as the number of IFN-y producing cells (spot forming cells, SFC) per million of spleen cells calculated for each group at day 24 post administration of the prime dose. Groups 5 (AdGP70 as the prime dose and PepGP70 as the boost dose) and 6 (PepGP70 as the prime dose and AdGP70 as the boost dose) are the heterologous prime boost dosing regimen groups showing a synergist effect as compared to the homologous prime boost groups (Groups 1 to 4). See Examples 1 and 3.

FIG. 2 shows the percentage of CD8+ T cells positive for dextramer staining at day 24 post administration of the prime dose Animal in Groups 1 to 4 were subjected to homologous prime boost doing regimen (AdGP70 or PepGP70) and the animals in Groups 4 and 5 were subjected to a heterologous prime boost doing regimen; AdGP70 as the prime dose and PepGP70 as the boost dose (Group 5) or PepGP70 as the prime dose and AdGP70 as the boost dose (group 6). See Example 3.

FIG. 3 shows the vaccine compositions PepGP70 and AdGP70, tested individually or as prime-boost combinations, synergize with an anti-PD1 treatment in their ability to promote an anti-tumor immune response, wherein antigen-specific CD8+ T cells were measured at day 6 (D6) and day 20 (D20). Animals in: Group 1 were administered PepGP70 (as the prime and boost dose)+anti-PD1; Group 2 were administered AdGP70 (as the prime and boost dose)+anti-PD1; Group 3 were administered AdGP70 as the prime dose and PepGP70 as the boost dose+anti-PD1; Group 4 were administered PepGP70 as the prime dose and AdGP70 as the boost dose+anti-PD1; Group 5 were administered only anti-PD1 (control); and, Group 6 were administered no treatment (control). AH1 peptide is the T cell epitope SPSYVYHQF (SEQ ID NO 72), which is present in GP70-472 and GP70-CM. See Examples 1, 2 and 5.

FIGS. 4A and 4B show the IFN-γ ELISpot response to the FP-OVA vaccine compared to the excipient group (FIG. 4A) and the anti-tumor activity in terms of overall survival of FP-OVA vaccine compared to the excipient against the E.G7-OVA tumor models (FIG. 4B). The animals in FIG. 4B were induced with E.G7-OVA cells at day 24, wherein the vaccine composition previously administered provided protection against death from tumor growth. See Example 6.

FIGS. 5A and 5B show the anti-tumor activity in terms of tumor growth prevention of FP-OVA vaccine (FIG. 5B) compared to the excipient (FIG. 5A) against the E.G7-OVA tumor models. The animals in FIG. 5B were induced with E.G7-OVA cells at day 24, wherein the vaccine composition previous administered at day 0 and day 14 provided protection against tumor growth. See Example 6.

FIG. 6 show the anti-tumor activity in terms of overall survival of FP-OVA vaccine (group 1 and 2) compared to the excipient (group 3) against the E.G7-OVA tumor models, wherein animals were administered the vaccine compositions after animals were induced with E.G7-OVA cells demonstrating the therapeutic effect of the vaccine composition.

FIG. 7 shows the vaccine compositions PepGP70 or AdGP70, tested individually or in combination with anti-PD1, synergize with an anti-PD1 treatment in their ability to promote an anti-tumor immune response, wherein tumor size was measured over a period of days. Group 2 (AdGP70+anti-PD1) provided 79% tumor free animals as compared to Group 1 (AdGP70) with only 43% tumor free animals; Group 4 (PepGP70+anti-PD1) provided 62% tumor free animals as compared to Group 3 (PepGP70) with only 15% tumor free animals. Anti-PD1 alone provided only 29% tumor free animals See Example 7.

FIGS. 8A and 8B shows overall survival for each group; Adenoviral vector GP70 (FIG. 8A) and Fluorocarbon linked GP70 peptides (FIG. 8B), with and without the immune checkpoint inhibitor anti-PD1. See Example 7.

### DETAILED DESCRIPTION OF THE INVENTION

Introduction

The present invention provides compositions and methods for inducing an enhanced T cell response. In particular, the instant vaccine combination induces T-cell mediated immunity, either prophylactically or therapeutically, in controlling persistent viral infections and cancer. The methods and compositions provided include administering a heterologous vaccine prime dose and boost dose leading to an induction of a T cell response where “heterologous” means a prime dose that is different than a boost dose, provided both comprise at least one, or more, of the same T-cell epitopes. In certain embodiments, that means a prime dose and boost dose wherein the antigen/immunogens/peptides are presented to the immune system via different delivery carriers and/or vectors. Applicants have found that administering different T cell inducing vaccines (e.g., viral vector encoding an antigen and micelles containing peptides that comprise shared T cell epitopes with the viral vector expressed antigen) synergistically boost inducing antigen specific T cell response. See Example 3. In embodiments, the prime dose and the boost dose comprise one or more of the same CD8+ T cell epitopes. In certain embodiments, the polypeptides of the prime dose and/or boost dose also comprise one or more CD4+ T cell epitopes.

The sequence of the antigen/immunogens may not be the same, but there must be overlap in at least the one or more T cell epitopes (e.g., one may be full length and the other a short peptide derived from the full-length antigen, including chimeric peptides) in the prime and boost dose. Those T cell epitopes can be identified in antigens or immunogens using well known techniques in the art, including from published references, including databases or websites that contain peptide sequences known to bind class I and/or class II MHC molecules complied from published reports (e.g. the SYFPEITHI website), or algorithms such as artificial neural network (ANN) or stabilized matrix method (SMM) (e.g., using the Immune Epitope Database and Analysis Resource website). See Example 1. In embodiments, the prime and boost dose comprise one or more shared CD8+ T cell epitopes.

In certain embodiments provided herein are vaccine combinations that comprise a first composition comprising a non-replicating viral vector encoding an antigen or immunogen containing one or more CD8+ T cell epitopes; and, a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen. In certain embodiments, the first composition is a prime dose and the second composition is the boost dose. In certain other embodiments, the first composition is the boost dose and the second composition is the prime dose.

As used herein, a “heterologous dosing regimen”, means a prime dose and boost dose wherein the antigen/immunogens/peptides are presented to the immune system via different delivery carriers and/or vectors. For example, in the instant invention a first composition (as either a prime dose or a boost dose) comprises a viral vectored antigen or immunogen, and a second composition (as either a prime dose or a boost dose) comprises fluorocarbon-linked peptides, wherein the peptide comprises one or more T cell epitopes in common with the viral vectored antigen or immunogen. In other words, in certain embodiments, the present vaccine combination is two different T cell inducing vaccine compositions, wherein each composition induces antigen specific CD8+ T cells against the same antigen.

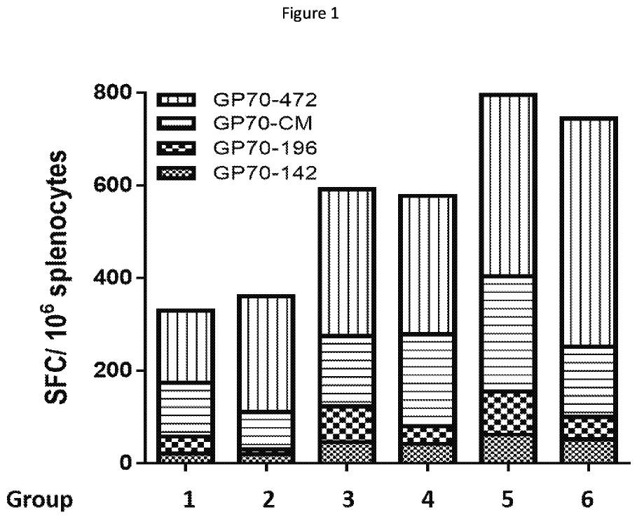

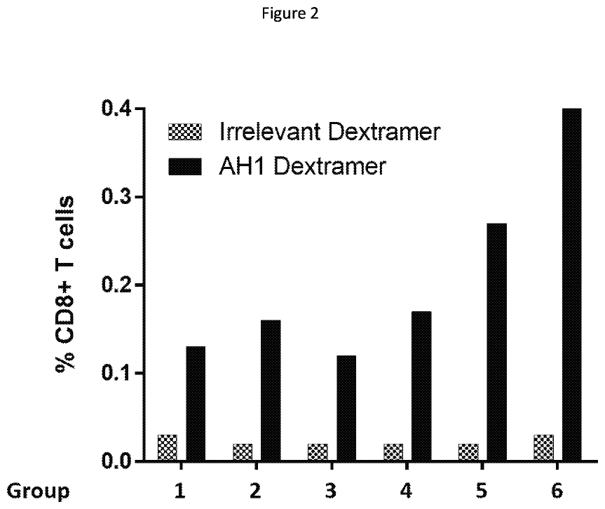

Applicants have found that a heterologous dosing regimen (wherein at least one prime dose and at least one boost dose are administered) with an adenoviral vectored antigen and a fluorocarbon-linked peptide provided a synergist effect of the induced immune response as compared to a homologous dosing regimen with either of the adenoviral vectored antigen or the fluorocarbon-linked peptide. See Example 3, and FIGS. 1 and 2. Groups 5 and 6 of FIGS. 1 and 2 represent the animals administered a heterologous dosing regimen, as compared to groups 1 to 4, which represent animals administered a homologous dosing regimen. Administration of a heterologous prime dose and boost dose induce an antigen specific CD8+ T cell response, as demonstrated in FIGS. 1 and 2.

In certain embodiments, the present vaccine combination is used as a therapeutic to treat tumors and/or prophylactic against cancer. The synergist effects of the present vaccine combinations may be used to treat certain tumors. See Example 4. In certain other embodiments, the present vaccine combination is used as a therapeutic to treat chronic infections.

In certain embodiments, the present vaccine combination further comprises a third composition comprising an immune modulator composition selected from an anti-immunosuppressor or an immunoactivator. Certain immune checkpoint proteins, such as PD1 on T cells and PD-L1 on tumor cells, help keep immune responses muted to those tumors, wherein when PD1 is bound by PD-L1 that interaction inhibits T cells from killing those tumor cells. Hence, blocking the interaction of certain immune checkpoint proteins with an anti-immunosuppressor allows the T cells to kill tumor cells. The instant invention combines the synergist effect of the heterologous dosing regimen to induce antigen specific CD8+ T cells, such as a cancer or viral antigen, specific for a tumor or cancer type (e.g., melanoma, lymphoma, lung cancer, etc.) with an anti-immunosuppressor, such as an immune checkpoint inhibitor that non-specifically signals T cells to kill cancer cells.

In embodiments, the anti-immunosuppressor is an MDSC inhibitor. Myeloid derived suppressor cells (MDSC) are a heterogenous group of immune cells derived from the myeloid lineage and which expand in certain pathologic conditions such as chronic infection and cancer; they may also play a role in certain autoimmune diseases. The MDSC possess strong immunosuppressive activities rather than immunostimulatory properties, wherein they interact with T cells and certain antigen presenting cells (APC) to regulate their function. MDSC are a known suppressor of T cells (both CD8+ and CD4+ T cells) and mediate tolerance induction in autoimmune diseases and cancer. Hence, inhibitors of MDSC activity can help break tolerance in certain disease states. The instant invention combines the synergist effect of the heterologous dosing regimen to induce antigen specific CD8+ T cells, such as a cancer or viral antigen, specific for a tumor or cancer type, with a myeloid derived suppressor cell (MDSC) inhibitor that helps break tolerance by inhibiting tolerogenic MSDC and allowing a more robust immune response by the induced CD8+ T cells.

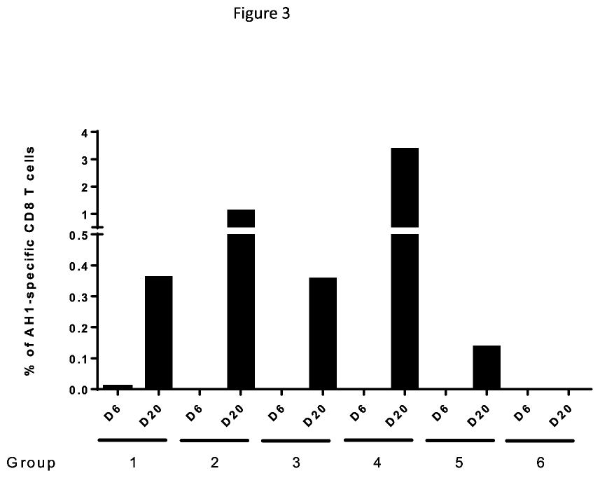

Applicants have found a synergist effect of the present heterologous dosing regimen in combination with the immune checkpoint inhibitor, anti-PD1, when the second composition (fluorocarbon linked peptide) is administered as the prime dose and the first composition (non-replicating viral vectored antigen or immunogen) is administered as the boost dose. See Example 5 and FIG. 3. In certain embodiments micelles containing fluorocarbon-linked peptides, wherein each peptide linked to the fluorocarbon is: i) 15 to 75 amino acid residues long; ii) from the antigen or immunogen of the boost dose; and, iii) contains one or more of the CD8+ T cell epitopes of the boost dose (e.g., PepGP70) is administered as the prime dose and a non-replicating viral vector encoding an antigen or immunogen containing one or more CD8+ T cell epitopes (e.g., AdGP70) is administered as the boost dose, in combination with administration of the immune checkpoint inhibitor anti-PD1. Fluorocarbon linked peptides are amphiphilic and spontaneously form micelles in certain solvents, wherein those micelles, when administered to an animal or human are preferentially taken up by antigen presenting cells (APC) for processing. See U.S. Pat. Nos. 7,687,455 and 9,119,811; the disclosures of each are herein incorporated by reference.

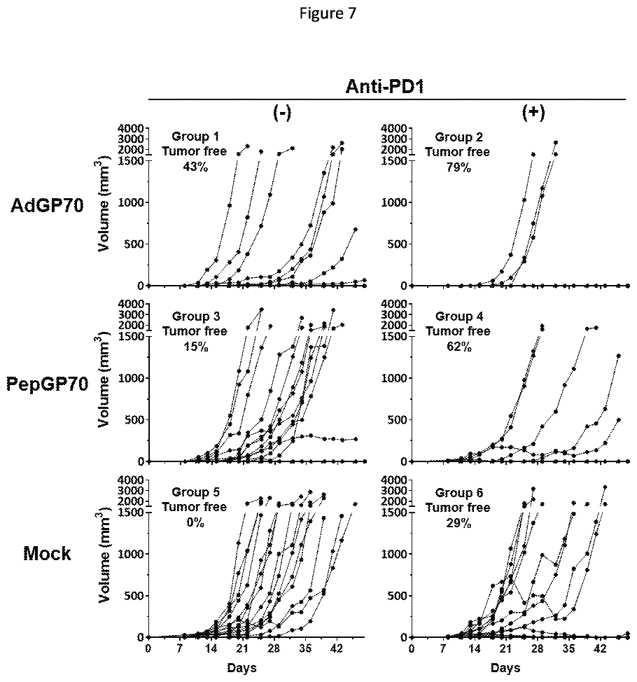

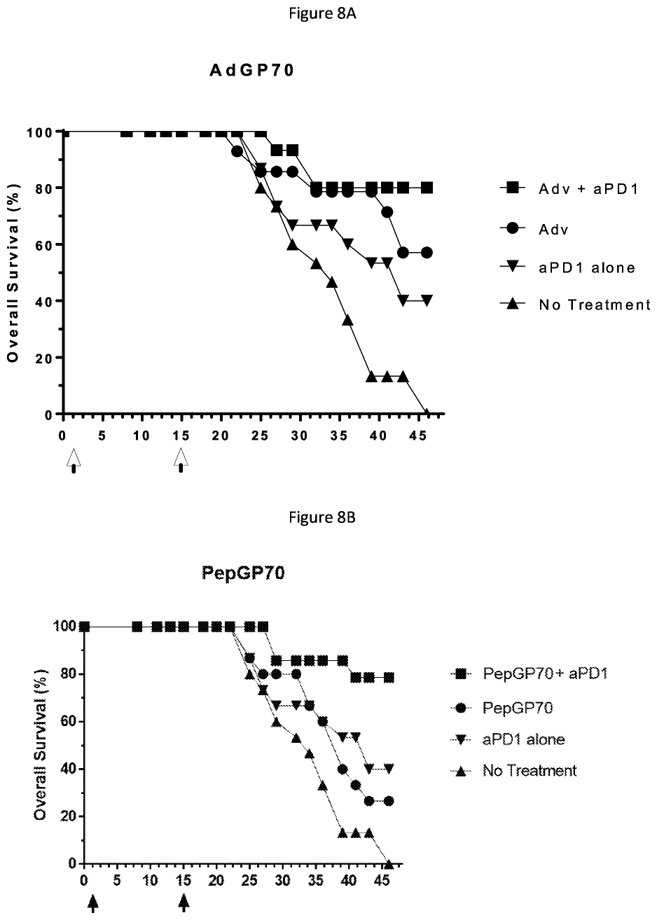

Applicants have found that administering either a first or second vaccine composition in combination with the third composition, an immune checkpoint inhibitor, provided a synergistic effect of the induced immune response as compared to administration of those compositions alone. See Example 7, and FIGS. 7, 8A and 8B. Groups 2 and 4 of FIG. 7 represent the animals administered the vaccine combination, as compared to groups 1 and 3, which represent animals administered either of the two T cell inducing vaccine compositions alone. Administration of the vaccine combination also increased the overall survival of the animals as compared to animals administered a single treatment, as demonstrated in FIGS. 8A and 8B.

In certain embodiments provided herein are vaccine combinations that comprise a first composition comprising a non-replicating viral vector encoding an antigen or immunogen containing one or more CD8+ T cell epitopes; and, a third composition comprising an immune modulator selected from an anti-immunosuppressor or an immunoactivator. In certain other embodiments, provided herein are vaccine combinations that comprise a second composition comprising micelles containing fluorocarbon-linked peptides, wherein each peptide linked to the fluorocarbon is: i) 15 to 75 amino acid residues long; ii) from an antigen or immunogen; and, iii) contains one or more of the CD8+ T cell epitopes of an antigen or immunogen; and, a third composition comprising an immune modulator selected from an anti-immunosuppressor or an immunoactivator.

In embodiments, the first composition comprises a non-replicating viral vector such as an adenoviral vector. Of all the replication-deficient viral vectors available, adenovirus is the most potent in priming T cell responses to the recombinant antigen. In embodiments, the second composition comprises a fluorocarbon linked peptide.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, an “adjuvant” refers to a substance that enhances the body's immune response to an antigen, in the present disclosure an adjuvant enhances the cell mediated immune response induced by the combination of the first composition, second composition and/or third composition. As used herein, an adjuvant is combined into any or all of the first composition, second composition and/or third composition. An adjuvant is distinct from the third composition of the present disclosure. Examples of adjuvants that may be used to enhance a cell mediate immune response, include, but are not limited to a Toll-like receptors (TLR) agonist: e.g. of agonist of TLR2, TLR3, TLR7, TLR8, or TLR9; or an agonist of STING, cGAS, NOD1, NOD2 or IRF3.

By “administration” is meant introducing a vaccine composition or combination of the present disclosure into a subject; it may also refer to the act of providing a composition of the present disclosure to a subject (e.g., by prescribing). The term “therapeutically effective amount” as used herein refers to that amount of the compound being administered which will induce a cell mediated immune response. The term also refers to an amount of the present compositions or combinations that will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in an animal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof. The term “effective amount” refers to that amount of the compound being administered which will produce a reaction that is distinct from a reaction that would occur in the absence of the compound. In reference to embodiments of the disclosure including the immunotherapy compounds of the disclosure, an “effective amount” is that amount which increases the immunological response in the recipient over the response that would be expected without administration of the compound.

The term “animal” refers to mammalian subjects, including humans, horses, dogs, cats, pigs, livestock, and any other mammal, along with birds. As referred to herein the term “animal” also includes an individual animal in all stages of development, including newborn, embryonic and fetal stages.

The term “host” or “organism” as used herein includes humans, mammals (e.g., cats, dogs, horses, etc.), insects, living cells, and other living organisms. A living organism can be as simple as, for example, a single eukaryotic cell or as complex as a mammal. Typical hosts to which embodiments of the present disclosure relate will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For research applications, a wide variety of mammals will be suitable subjects, including rodents (e.g., mice, rats, hamsters), rabbits, primates, and swine such as inbred pigs and the like. Additionally, for in vitro applications, such as in vitro research applications, body fluids and cell samples of the above subjects will be suitable for use, such as mammalian (particularly primate such as human) blood, urine, or tissue samples, or blood, urine, or tissue samples of the animals mentioned for veterinary applications. Hosts that are “predisposed to” condition(s) can be defined as hosts that do not exhibit overt symptoms of one or more of these conditions but that are genetically, physiologically, or otherwise at risk of developing one or more of these conditions.

The terms “protein,” “polypeptide,” and “peptide” may be referred to interchangeably herein. However, the terms may be distinguished as follows. A “protein” typically refers to the end product of transcription, translation, and post-translation modifications in a cell. As used herein a “polypeptide” can refer to a “protein” or a “peptide”. A “peptide”, in contrast to a “protein”, typically is a short polymer of amino acids, of a length typically of 100 or less amino acids.

The compositions, formulations and methods of the present invention may comprise, consist essentially of, or consist of the components and ingredients of the present invention as well as other ingredients described herein. As used herein, “consisting essentially or means that the compositions, formulations and methods may include additional steps, components or ingredients, but only if” the additional steps, components or ingredients do not materially alter the basic and novel characteristics of the claimed compositions, formulations and methods.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

As used herein, the term “human adenovirus” is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus may be of serogroup A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

As used herein “immune modulator” refers to a range of treatments aimed at harnessing a patient's immune system to achieve immune control, stabilization, and potential eradication of disease. For example, an immune modulator may be a substance used to break tolerance (such as may be present in a chronic infection or certain autoimmune diseases); or a substance used to inactivate immune suppressor cells to induce or enhance a host cell mediated immune response against a foreign or self (e.g., cancer) antigen; or an immunostimulatory substance also used to induce or enhance a host cell mediated immune response against a foreign or self (e.g., cancer) antigen. In embodiments, immune modulators comprise a substance that modulates T-cell pathways and have the potential to reinvigorate an antitumor or antiviral immune response.

A “pharmaceutical composition” refers to a mixture of one or more of the vaccine compounds described herein, derivatives thereof, or pharmaceutically acceptable salts thereof, with other chemical components, such as pharmaceutically acceptable carriers and excipients. One purpose of a pharmaceutical composition is to facilitate administration of a compound to the organism.

As used herein, a “pharmaceutically acceptable carrier” refers to a carrier or diluent that does not cause significant irritation to an organism and does not abrogate the biological activity and properties of the administered vaccine compositions.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also mean prolonging survival as compared to expected survival if not receiving treatment and/or can be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating” and refers to the act of increasing the time before the actual onset of a condition in a patient that is predisposed to the condition.

As referred to herein, a “vaccine” can include an antigen or vector, along with other components of a vaccine formulation, including for example adjuvants, slow release compounds, solvents, etc. Although vaccines are traditionally used to prevent or treat infectious diseases, vaccines are also able to modify the function of metabolites by binding signaling peptides or proteins or their receptors and by blocking antigens unique to certain abnormal cell types, such as for example, tumors. Accordingly, it is an embodiment of the invention provide vaccines to improve immune response to any antigen regardless of the antigen source or its function, including antigens to alter physiological functions that are desirable to improve health, such as immunizing against cancer.

As referred to herein, a “vector” carries a genetic code, or a portion thereof, for an antigen, however it is not the antigen itself. In an exemplary aspect, a vector can include a viral vector or bacterial vector. As referred to herein an “antigen” means a substance that induces a specific immune response in a subject, including humans and/or animals. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, or any combination thereof. In various aspects, the antigen is a virus, bacterium, a subunit of an organism, an auto-antigen, or a cancer antigen.

Vaccine Combinations

Provided herein are vaccine combinations that comprise a first composition and a second composition, wherein those compositions serve as a heterologous prime dose and a boost dose for inducing T-cell mediated immunity. The first composition and second composition comprise one or more CD8+ T cell epitopes that are the same. In certain embodiments, the first or second composition may comprise a full-length antigen or immunogen, or a fragment thereof, each of which are also referred to herein as a “polypeptide”. In certain embodiments, the prime dose (as either a first or second composition) comprises a full-length antigen or immunogen, and the boost dose (as either a first or second composition) comprises a fragment thereof such as a peptide, including a chimeric peptide. In certain other embodiments, the prime dose (as either a first or second composition) comprises a fragment of an antigen or immunogen, such as a peptide, including chimeric peptides, and the boost dose (as either a first or second composition) comprises a full-length antigen or immunogen. In certain other embodiments, the prime dose (as either a first or second composition) comprises a fragment of an antigen or immunogen, such as a peptide, including chimeric peptides, and the boost dose (as either a first or second composition) comprises a fragment of an antigen or immunogen, such as a peptide, including chimeric peptides. Heterologous, as used herein does not refer to the antigen or immunogen, but to the delivery vector or carrier linked to the antigen or immunogen.

In embodiments, the first and second compositions comprise a delivery vector or carrier, which may include peptides, lipophilic chains, or expression vectors including non-replicating viral vectors. In certain embodiments, the carrier may be a hydrocarbon chain, optionally substituted with one or more halogen atoms. In certain embodiments, the carrier may be a fluorocarbon chain. In certain embodiments the delivery vector may be a non-replicating viral vector, such as those selected from an adenovirus vector, an alphavirus vector, a herpesvirus vector, a measles virus vector, a poxvirus vector, or a vesicular stomatitis virus vector. In certain embodiments, the non-replicating viral vector is an E1 and/or E3 deleted adenovirus vector. In certain embodiments the non-replicating adenoviral vector is a human adenoviral vector.

Provided herein are vaccine combinations comprising a first composition and a second composition, wherein the first composition comprises a non-replicating viral vector encoding an antigen or immunogen containing one or more CD8+ T cell epitopes and the second composition comprises micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen. Either of the first or second compositions may be the prime or boost dose. To provide a heterologous dosing regimen, each of the first and second compositions must be administered to an animal in need thereof, one as the prime dose and the other as a boost dose.

Further embodiments provided herein are vaccine combinations that comprise a first composition or a second composition and a third composition, wherein the first or second composition induce T cell immunity and the third composition is an immune modulator selected from an anti-immunosuppressor or an immunoactivator, that in combination enhance the antigen specific T cell response. The first composition and second composition comprise one or more CD8+ T cell epitopes. In certain embodiments, the first or second composition may comprise a full-length antigen or immunogen, or a fragment thereof, each of which are also referred to herein as a “polypeptide”. In certain embodiments, first composition comprises a full-length antigen or immunogen. In certain other embodiments, the first composition comprises a fragment of an antigen such as a peptide, including a chimeric peptide. In certain embodiments, second composition comprises a full-length antigen or immunogen. In certain other embodiments, the second composition comprises a fragment of an antigen or immunogen, such as a peptide, including chimeric peptides.

Provided herein are vaccine combinations comprising a first composition or a second composition and a third composition, wherein the first composition comprises a non-replicating viral vector encoding an antigen or immunogen containing one or more CD8+ T cell epitopes; the second composition comprises micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes from the antigen or immunogen; and the third composition comprises an immune modulator selected from an anti-immunosuppressor or an immunoactivator.

Antigen and Immunogens

The choice of antigen or immunogen (which may be used herein interchangeably) is determined by whether T cell responses against the antigen have been found to be protective and/therapeutic or are expected to be protective and/or therapeutic, either in humans or animal models. The degree of polymorphism found between different isolates of antigen as well as highly conserved antigens, polymorphisms of HLA molecules, or highly conserved epitopes within antigens, are also factors in selecting antigens. See U.S. Pat. No. 8,642,531 and US Pat. Publ. No. 2016/0106830 for T cell vaccine compositions based on highly conserved antigen T cell epitopes and polymorphism of HLA molecules across different population and/or ethnic groups of people, the content of which are herein incorporated by reference. Also see, US Pat. Publ. No. 2016/0199469 at paragraphs [0121] to [0134], the content of which is herein incorporated by reference. In certain embodiments, antigens may also be selected that are a combination of conserved regions, or conserved epitopes, from one or more antigens to produce a synthetic vaccine antigen [Goodman A L, et al. New candidate vaccines against blood-stage Plasmodium falciparum malaria: prime-boost immunization regimens incorporating human and simian adenoviral vectors and poxviral vectors expressing an optimized antigen based on merozoite surface protein 1. Infection and Immunity 2010; 78(11):4601-12; Letourneau S, et al. Design and pre-clinical evaluation of a universal HIV-1 vaccine. PLoS ONE 2007; 2(10):e9841. In certain embodiments, the combination may be in the form of a pool of peptides or polypeptides in a first or second vaccine composition, or as a chimeric peptide or polypeptide in the first or second vaccine composition. In certain embodiments, the first and second vaccine compositions comprise more than one antigen to reduce the likelihood of immune escape. See US Pat. Publ. No. 2016/0199469 for a T cell oncology vaccine comprising two or more antigens, the content of which is herein incorporated by reference.

T cell epitopes within the antigens may be identified either by bioinformatics prediction and experimental confirmation, or by taking an empirical approach using a library of peptides spanning the complete antigen sequence. See Example 1 for more disclosure on identification of T cell epitopes (both CD8+ and CD4+). However, although knowledge of epitopes presented by common HLA types may be helpful in conducting detailed phenotypic studies of T cell responses in clinical research and vaccine development, a complete knowledge of all possible epitopes contained within the antigen is not necessary. The instant vaccine compositions, first and/or second compositions, comprise at least one CD8+ T cell epitope. In certain embodiments, the first and second compositions comprise at least two, three, four, five or at least six CD8+ T cell epitopes. In certain other embodiments, the first and/or second vaccine composition further comprise one or more CD4+ T cell epitopes. It is understood, the antigens in the form of polypeptides in the vaccine compositions may comprise one or more T cell epitopes that are not identified using the tools available for identification.

In embodiments, the antigen/immunogen may correspond to a smaller portion or polypeptide derived from a full-length antigen sequence that may be selected based on sequence conservation and/or the presence of CD8+ and CD4+ T cells epitopes. CD4+ and/or CD8+ T cell epitopes may be identified using bioinformatics tools identifying HLA class I and/or HLA class II binding sequences, in vitro assays using human PBMC samples or in vitro HLA class I and/or HLA class II binding assays. The immunogen may be generated by artificial concatenation of smaller sequence portions or polypeptides derived from a single or multiples antigen sequences from the same pathogen. The concatenate sequences may be used for the generation of synthetic peptide immunogens or the corresponding DNA sequence may be incorporated into an adenoviral vector.

In certain embodiments, the antigen or immunogen is from a pathogen, a cancer antigen or an auto-antigen. In certain embodiments, the antigen or immunogen is a neoantigen. As used herein, “neoantigen” refers to a known host protein with one or more amino acid changes as compared to wild type that allows the host immune system to recognize as foreign. Neoantigens may be unique to each patient and therefor, as used herein, may be referred to as protein variants including clinical variants identified from individual patients. Human clinical variants can be identified using well known databases that provide a public archive of reports of the relationships among human variations and phenotypes. Landrum M J, et al. ClinVar: public archive of interpretations of clinically relevant variants. Nucleic Acids Res. 2016 Jan. 4; 44(D1):D862-8. These neoantigens or clinical variants are mapped for CD8+ T cell epitopes that then allow for the development of the present vaccine combinations. Disclosure as to using well known tools for mapping T cell epitopes of antigens or immunogens is provided in Example 1.

In certain embodiments, the antigen or immunogen is from a pathogen, including those known to lead to cancer development (and thus may be expressed on the surface of cancer cells). In embodiments, the pathogen is a virus, fungus, parasite, or bacteria. In certain embodiment, the antigen or immunogen are from the virus selected from EBV, HPV, HTLV-1, MCPvV, KSHV or HERV. Each of these viruses are known to induce cancer or tumor growth. In certain embodiments, the antigen or immunogen are from the virus of HCV (Hepatitis C virus) or HBV (Hepatitis B virus).

In certain embodiments, the antigen or immunogens may be any one or more of the following: EBV (EBNA1, LMP1, LMP2 and BARF1), HPV (E1 to E7), HTLV-1 (tax), MCPyV (large T, small T), KSHV (Orf73, Orf57, K10.5, and K12), CMV (glycoprotein B and phosphoprotein 65), BKV ((large T, small T), JCV (large T, small T), SV40 (large T, small T), HERV (Env, Gag, Pol), HMTV (Env, Gag, Pol), HIV-1 (Env, Gag, Pol, Nef, Tat, Vif), HCV (Core, NS3, NS4, NS5), HBV (Core, Pol, Env and X), Influenza (HA, NP, NA, Ml, PB1, PB2, PA), HRV (VP1-4, 2A-C, 3A-D), Mycobacterium tuberculosis (Ag85A, ASAT-6, ROE. CFP-10) and Plasmodium falciparum (MSP-1. AMA-1, RTS,S, Pfs25).

In embodiments, the antigen induces an immune response against pathogens, including for example a virus. Exemplary viruses include an orthomyxovirus, a paramyxovirus, a rhinovirus, coronavirus, influenza virus, respiratory syncytial virus (RSV), a common cold virus or measles virus, herpes virus, rabies virus, varicella, human papilloma virus (HPV), hepatitis virus, or other known viral pathogens.

In embodiments, the antigen induces an immune response against bacterial pathogens. Exemplary bacteria include Bacillus, Mycobacterium, Staphylococcus, Streptococcus, Pseudomonas, Klebsiella, Haemophilus, Mycoplasm and/or Bacillus anthracis. In certain embodiments, the antigen induces an immune response against a fungal pathogen. In embodiments, the fungus includes Aspergillus, Candida, Cryptococcus, Histoplasma, Pneumocystis, and/or Stachybotrys.

In embodiments, the antigen can include an allergen, or a tumor associated antigen. In a still further aspect, the antigen may include polypeptides, peptides, or panels thereof that comprise one or more epitopes of a protein associated with a disease. For example, suitable polypeptides, peptides, or panels thereof may comprise one or more epitopes of a protein associated with a pathogen. Suitable polypeptides may comprise the full-length amino acid sequence of a corresponding protein of a pathogen or a fragment thereof.

First Composition of the Vaccine Combination

In embodiments, the first composition comprises a non-replicating viral vector encoding an antigen or immunogen containing one or more CD8+ T cell epitopes. In certain embodiments, the non-replicating viral vector is an adenovirus vector, an alphavirus vector, a herpesvirus vector, a measles virus vector, a poxviruses vector, or a vesicular stomatitis virus vector. In exemplary embodiments, the non-replicating viral vector is an adenoviral vector. Deletion or disruption of the E1 and/or E3 sequence results in a non-replicating adenoviral vector.

In embodiments, the compositions and formulations include a vector, namely a viral vector. As referred to herein, a “viral vector” is an engineered virus that incorporated genes for and express an antigen. Viral vectors may be non-replicating and are safe for the host and environment. It should be appreciated that any viral vector may be incorporated into the compositions, formulations and methods of the invention to effectuate delivery into a cell.

Exemplary viral vectors include adenovirus, retrovirus, lentivirus, herpes virus, pox virus, alpha virus, adeno-associated viruses, among others. Many such viral vectors are available in the art. The vectors described herein may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, Calif.).

In certain embodiments, the non-replicating viral vector is an adenovirus, including for example wherein the adenovirus is a bovine adenovirus, a canine adenovirus, a non-human primate adenovirus, a chicken adenovirus, or a porcine or swine adenovirus. In certain embodiments, the non-replicating viral vector is a human adenovirus.

In embodiments, non-replicating adenoviral vectors are particularly useful for gene transfer into eukaryotic cells and vaccine development, and in animal models.

In embodiments, any adenoviral vector (Ad-vector) known to one of skill in art, and prepared for administration to a mammal, which may comprise and express an antigen or immunogen may be used in the compositions and with the methods of this application. Such Ad-vectors include any of those in U.S. Pat. Nos. 6,706,693; 6,716,823; 6,348,450; or US Patent Publ. Nos. 2003/0045492; 2004/0009936; 2005/0271689; 2007/0178115; 2012/0276138 (herein incorporated by reference in entirety).

In certain embodiments the recombinant adenovirus vector may be non-replicating or replication-deficient requiring complementing E1 activity for replication. In embodiments the recombinant adenovirus vector may include E1-defective, E3-defective, and/or E4-defective adenovirus vectors, or the “gutless” adenovirus vector in which viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. In embodiments, the recombinant adenovirus vector is an E1 and/or E3 defective vector.

The “gutless” adenovirus vector replication requires a helper virus and a special human 293 cell line expressing both E1a and Cre, a condition that does not exist in natural environment; the vector is deprived of viral genes, thus the vector as a vaccine carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant may be constructed by cloning specific transgenes or fragments of transgenes into any of the adenovirus vectors such as those described below. The adenovirus recombinant vector is used to transduce epidermal cells of a vertebrate in a non-invasive mode for use as an immunizing agent. The adenovirus vector may also be used for invasive administration methods, such as intravenous, intramuscular, or subcutaneous injection.

In embodiments, the first composition comprising the non-replicating viral vector expressing the antigen or immunogen of the vaccine combination of interest may be formulated for administration to a mammal. With respect to dosages, routes of administration, formulations, adjuvants, and uses for recombinant viruses and expression products therefrom, compositions of the invention may be used for parenteral or mucosal administration, preferably by intradermal, subcutaneous, intranasal or intramuscular routes. When mucosal administration is used, it is possible to use oral, ocular or nasal routes.

The formulations which may comprise the adenovirus vector of interest, can be prepared in accordance with standard techniques well known to those skilled in the pharmaceutical or veterinary art. Such formulations can be administered in dosages and by techniques well known to those skilled in the clinical arts taking into consideration such factors as the age, sex, weight, and the route of administration. The formulations can be administered alone or can be co-administered or sequentially administered with compositions, e.g., with “other” immunological composition, or attenuated, inactivated, recombinant vaccine or therapeutic compositions thereby providing multivalent or “cocktail” or combination compositions of the invention and methods employing them. In embodiments, the formulations may comprise sucrose as a cryoprotectant and polysorbate-80 as a non-ionic surfactant. In certain embodiments, the formulations further comprise free-radical oxidation inhibitors ethanol and histidine, the metal-ion chelator ethylenediaminetetraacetic acid (EDTA), or other agents with comparable activity (e.g block or prevent metal-ion catalyzed free-radical oxidation).

The formulations may be present in a liquid preparation for mucosal administration, e.g., oral, nasal, ocular, etc., formulations such as suspensions and, preparations for parenteral, subcutaneous, intradermal, intramuscular, intravenous (e.g., injectable administration) such as sterile suspensions or emulsions. In such formulations the adenoviral vector may be in admixture with a suitable carrier, diluent, or excipient such as sterile water, physiological saline, or the like. The formulations can also be lyophilized or frozen. The formulations can contain auxiliary substances such as wetting or emulsifying agents, pH buffering agents, adjuvants, preservatives, and the like, depending upon the route of administration and the preparation desired. The formulations can contain at least one adjuvant compound.

Standard texts, such as “REMINGTON'S PHARMACEUTICAL SCIENCE”, 17th edition, 1985, incorporated herein by reference, may be consulted to prepare suitable preparations, without undue experimentation.

Second Composition of the Vaccine Combination

In embodiments, the second composition comprises micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the antigen or immunogen.

In embodiments, the peptide has a length from 20 to 60 amino acids, such as from 25 to 50 amino acids, from 30 to 40 amino acids, for example, 31, 32, 33, 34, 35, 36, 37, 38, 39, 40, 41, 42, 43, 44 or 45 amino acids. In certain embodiments, the peptide may include additional short amino acid sequences. The additional sequences may facilitate manufacture or formulation of the peptide or enhance stability of the peptide. For example, the peptide may comprise one or more additional amino acids, typically at the N-terminus and/or the C-terminus to enhance the net positive charge of the peptide and/or to reduce the hydrophobicity of the peptide. The net positive charge may be increased so that the peptide has an isoelectric point greater than or equal to 7.

In certain embodiments, one or more, such as two or three positively charged amino acids (arginine and/or lysine) are added to the N- and/or C-terminus of one or more of the peptides in the composition. For example, three lysine residues (KKK) may be added to the N- and/or C-terminus of one or more of the peptides. See Table 2. Positive amino acids are typically added at the end(s) of peptides that have an overall hydrophobicity of more than 65%, a net charge of less than zero and/or include cluster of hydrophobic amino acids.

In embodiments, where the peptide is linked to a fluorocarbon, the terminus of the peptide, such as the terminus that is not conjugated to the fluorocarbon, or other attachment, can be altered, for example to promote solubility of the fluorocarbon-peptide construct via the formation of micelles. To facilitate large-scale synthesis of the construct, the N- or C-terminal amino acid residues of the peptide can be modified. When the desired peptide is particularly sensitive to cleavage by peptidases, the normal peptide bond can be replaced by a non-cleavable peptide mimetic. Such bonds and methods of synthesis are well known in the art.

The peptide may be a native peptide. The native peptide may have free or modified extremities. The peptide may be modified to increase longevity, such as half-life or persistence at the site of administration, of the peptide in vivo or to direct the peptide to antigen-presenting cells. For example, the immunogenic peptide can contain one or more non-naturally occurring amino acids and/or non-naturally occurring covalent bonds for covalently connecting adjacent amino acids. In certain embodiments, the non-standard, non-naturally occurring amino acids can also be incorporated into the immunogenic peptides provided that they do not interfere with the ability of the peptide to interact with HLA molecules and remain cross-reactive with T-cells recognizing the natural sequences. Non-natural amino acids can be used to improve peptide resistance to protease or chemical stability. Examples of non-natural amino acids include D-amino acids and cysteine modifications.

In embodiments, the second composition may comprise multiple peptides linked to fluorocarbon chains. Accordingly, the composition may comprise at least two, such as at least three, four, five, six, seven, eight, nine, ten, eleven, twelve, thirteen, fourteen, or more peptides. In exemplary embodiments, the second composition comprises four peptides, each of which are linked to a fluorocarbon chain. See Example 1.

Whether or not a peptide is able to induce a peptide specific response in T cells of a cancer patient, a patient with a chronic infection, or a healthy subject may be determined by any suitable means, typically by testing a sample of peripheral blood mononucleated cells (PBMCs) taken from said patient or subject in a suitable assay. The T cell response is thus detected in vitro in the sample. Suitable assays may measure or detect the activation of T cells following incubation with a test peptide. Activation of T cells may typically be indicated by the secretion of a cytokine, such as IFN-gamma, which may be detected in any suitable assay, typically an immunoassay such as an ELISA or ELISpot. The magnitude of the T cell response of a patient or subject may be determined in the same assay, for example by quantifying the amount of cytokine released in the sample as a whole, or by a particular cell in the sample, following incubation with a test peptide. Suitable assays are described further below and in the Examples.

In certain embodiments, the second composition of the invention comprises a peptide or peptides that induces a specific CD8+ T cell response in at least 20% of healthy subjects and/or cancer patients Immunological assays for measuring peptide-specific T cell responses in human peripheral blood mononuclear cells (PBMCs) from healthy subjects or cancer patients may be carried out by mean of cytokine ELISpot, such as the IFN-γ ELISpot assay or intracellular cytokine staining using flow cytometry. The assays may be performed either from fresh or frozen PBMCs. The assays may be performed either ex vivo or after short term in vitro cultures of PBMCs incubated with a single peptide or a composition comprising several antigenic peptides. The amount of the peptide(s) in the short term in vitro culture may vary from 0.001 μg per peptide to 100 μg/peptide. The incubation time for short term in vitro cultures may be between 5 to 15 days, such as 7 to 13 days or 9 to 11 days. Short term in vitro cultures may be performed in the presence of cytokines, such as one or more of IL-2, IL-15 and IL-7, preferably IL-2 and IL-15. Short term in vitro cultures can be performed after depletion of T regulatory cells and/or NK cells. Depletion of such cells may be particularly desirable when the PBMC are from a cancer patient. Short term in vitro culture may be performed in the presence of IL-10 neutralizing antibodies, anti-PD1 antibodies, anti-CTLA-4 antibodies, anti-OX-40 antibodies, anti-GITR antibodies, denileukin, diftitox, kinase inhibitors and/or toll receptor agonists including agonists of TLR2, TLR3, TLR7, TLR8 and TLR9.

In certain embodiments, the carrier is a hydrocarbon chain substituted with one or more halogen atoms. In embodiments, the carrier is a hydrocarbon chain substituted with one or more fluorine atoms, herein referred to as a “fluorocarbon chain”.

The fluorocarbon can comprise one or more chains derived from perfluorocarbon or mixed fluorocarbon/hydrocarbon radicals, and may be saturated or unsaturated, each chain having from 3 to 30 carbon atoms. Thus, the chains in the fluorocarbon attachment are typically saturated or unsaturated, preferably saturated. The chains in the fluorocarbon attachment may be linear or branched, but preferably are linear. Each chain typically has from 3 to 30 carbon atoms, from 5 to 25 carbon atoms, or from 8 to 20 carbon atoms. In order to covalently link the fluorocarbon vector to the peptide, a reactive group, or ligand, for example —CO—, —NH—, S, O or any other suitable group is included in the vector. The use of such ligands for achieving covalent linkages is well known in the art. The reactive group may be located at any position on the fluorocarbon vector.

Coupling of the fluorocarbon vector to the peptide may be achieved through functional groups such as —OH, —SH, —COOH and —NH₂, naturally present or introduced onto any site of the peptide. Examples of such linkages include amide, hydrazone, disulphide, thioether and oxime bonds.

Optionally, a spacer element (peptidic or non-peptidic) can be incorporated to permit cleavage of the peptide from the fluorocarbon element for processing within an antigen-presenting cell and to optimize steric presentation of the peptide. The spacer can also be incorporated to assist in the synthesis of the molecule and to improve its stability and/or solubility. Examples of spacers include polyethylene glycol (PEG) or amino acids such as lysine or arginine that may be cleaved by proteolytic enzymes.

In certain embodiments, the fluorocarbon-linked peptide can have the chemical structure CₘFₙ—C_{y}Hₓ-(Sp)-R or derivatives thereof, where m=3 to 30, n≤2m+1, y=0 to 15, x≤2y, (m+y)=3 to 30 and Sp is an optional chemical spacer moiety and R is an immunogenic peptide. Typically m and n satisfy the relationship 2m−1≤n≤2m+1, and preferably n=2m+1. Typically x and y satisfy the relationship 2y−2≤x≤2y, and preferably x=2y. Preferably the cₘFₙ-c_{y}Hₓ moiety is linear.

In embodiments, m is from 5 to 15, more preferably from 8 to 12. In other embodiments, y is from 0 to 8, more preferably from 0 to 6 or 0 to 4. In embodiments, the CₘFₙ—C_{y}Hₓ moiety is saturated (i.e., n=2m+1 and x=2y) and linear, and that m=8 to 12 and y=0 to 6 or 0 to 4.

In certain embodiments, the fluorocarbon vector is derived from 2H, 2H, 3H, 3H-perfluoroundecanoic acid of the following formula:

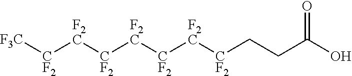 [SMILES: O=C(O)CCC(F)(F)C(F)(F)C(F)(F)C(F)(F)C(F)(F)C(F)(F)C(F)(F)C(F)(F)F]

In embodiments, the fluorocarbon attachment is the linear saturated moiety C₈F₁₇(CH₂)₂— which is derived from C₈F₁₇(CH₂)₂COOH. In certain embodiments, the fluorocarbon attachments have the following formulae: C₆F₁₃(CH₂)₂—, C₇F₁₅(CH₂)₂—, C₀F₁₀ (CH₂)₂—, C₁₀F₂₁ (CH₂)₂—, C₅F₁₁(CH₂)₃—, C₆F₁₃ (CH₂)₃—, C₇F₁₅ (CH₂)₃—, C₈F₁₇(CH₂)₃— and C₉F₁₉(CH₂)₃— which are derived from C₆F₁₃(CH₂)₂COOH, C₇F₁₅(CH₂)₂COOH, C₉F₁₉(CH₂)₂COOH, C₁₀F₂₁(CH₂)₂COOH, C₅F₁₁(CH₂)₃COOH, C₆F₁₃(CH₂)₃COOH, C₇F₁₅(CH₂)₃COOH, C₈F₁₇(CH₂)₃COOH and C₉F₁₉(CH₂)₃COOH respectively.

In embodiments, examples of suitable structures for the fluorocarbon vector-antigen constructs have the formula:

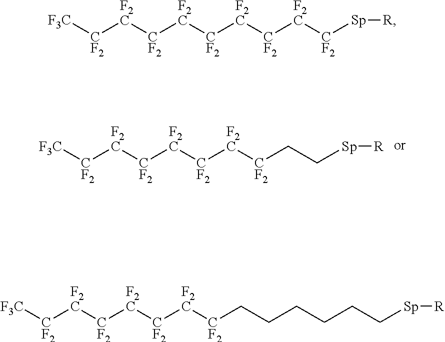

in which Sp and R are as defined above. In certain embodiments Sp is derived from a lysine residue and has the formula —CONH—(CH₂)₄—CH(NH₂)—CO—. In embodiments, R is a peptide that is 15 to 75 amino acid residues long; ii) from the antigen or immunogen of the first composition; and, iii) contains one or more of the CD8+ T cell epitopes of the first composition from the antigen or immunogen.

In certain embodiments, the fluorocarbon attachment may be modified such that the resulting compound is still capable of delivering the peptide to antigen presenting cells. Thus, for example, a number of the fluorine atoms may be replaced with other halogen atoms such as chlorine, bromine or iodine. In addition, it is possible to replace a number of the fluorine atoms with methyl groups or hydrogen and still retain the properties of the molecule described herein.

In embodiments, the peptides may be linked to the fluorocarbon vector via a spacer moiety. In one embodiment the spacer moiety is a lysine residue. This spacer residue may be present in addition to any terminal lysine residues as described above, so that the peptide may, for example, have a total of four N-terminal lysine residues. Accordingly, in certain embodiments, the second composition of the invention may comprise fluorocarbon-linked peptides in which the peptides have a C-terminal or N-terminal lysine residue, preferably an N-terminal lysine residue. In embodiments, the terminal lysine in the peptides is linked to a fluorocarbon having the formula C₈F₁₇ (CH₂)₂COOH. In embodiments, the fluorocarbon is coupled to the epsilon chain of the N-terminal lysine residue.

In embodiments, the second composition comprises at least 1, 2, 3, 4, 5, 6, 7, 8, 9 or 10 or more immunogenic peptides linked to its own fluorocarbon vector.

The Third Composition of the Vaccine Combination

In embodiments, the third composition comprises an immune modulator. Immune modulators comprise a range of treatments (e.g. substances or compounds) aimed at harnessing a patient's immune system to achieve immune control, stabilization, and potential eradication of disease.

In embodiments, immune modulators comprise immune checkpoint-blocking antibodies that modulate T-cell pathways and have the potential to reinvigorate an antitumor or antiviral immune response. In certain embodiments, immune checkpoint inhibitors include, but are not limited to, Ipilimumab, the first FDA-approved immune checkpoint antibody licensed for the treatment of metastatic melanoma that blocks a checkpoint molecule called cytotoxic T-lymphocyte antigen 4 (CTLA-4) and other compounds (e.g. antibodies) targeting co-inhibitory receptors such as CTLA-4, PD-1, Lag-3, Tim-3, TIGIT and/or Vista.

In embodiments, immune modulators comprise substances targeting co-stimulatory receptors expressed by T cells such as those selected from tumor necrosis factor receptors (TNFRs), including and not limited to glucocorticoid-induced TNFR (GITR; CD357), CD27, OX40 (CD134), ICOS (CD278) or 4-1BB (CD137) where ligation of these glycoproteins with agonist antibodies actively conveys activating signals to the lymphocyte.

In certain embodiments, immune modulators comprise inhibitors of suppressive myeloid cells such as, for example, PDL1, PDL2, VISTA, B7-1, CD47, CD200, GLA1, GAL3, CLECG4 or SIRPa. In embodiments, immune modulators comprise compounds targeting a range of Toll-like receptors (TLR) and NOD-like receptors (NLR) that represent targets for a class of agonist but also antagonist molecules. In further embodiments, immune modulators comprise cytokines that are known to modulate T cell responses such as granulocyte colony-stimulating factor (G-CSF), interferons, IL-2, IL-7, or IL-12.

In embodiments, the third composition comprises an immune checkpoint inhibitor that targets PD1, PDL1, PDL2, CD28, CD80, CD86, CTLA4, B7RP1, ICOS, B7RPI, B7-H3, B7-H4, BTLA, HVEM, KIR, TCR, LAG3, CD 137, CD137L, OX40, OX40L, CD27, CD70, CD40, CD40L, TIM3, GALS, ADORA, CD276, VTCN1, IDO1, KIR3DL1, HAVCR2, VISTA or CD244. See e.g. Example 5 which provides a method for targeting PD1.

In embodiments, the third composition comprises an immune checkpoint inhibitor selected from Ipilimumab, Nivolumab, Pembrolizumab, Atezolizumab, Avelumab, Durvalumab, Cemiplimab, REGN2810, BMS-936558, SHR1210, KN035, IBI308, PDR001, BGB-A317, BCD-100, or JS001.

In other embodiments, the third composition comprises a MDSC inhibitor that targets ADAM17, PEG-2, PDE5, COX2, iNOS2, PDE5, c-kit, ARG1, PI3K, CSF-1R, Caspase-8, CCL2, RON, ROS, S100A8/A9 or liver-X nuclear receptor.

In embodiments, the MDSC inhibitor is PF-5480090, INCB7839, nitro-aspirine, SC58236, Celecoxib, IPI-549, PLX3397, BLZ945, GW2580, RG7155, IMC-CS4, AMG-820, ARRY-382, sildenafil, tadalafil, vardenafil, N-hydroxy-nor-L-Arg, imatinib, z-IETD-FMK, trabectedin, Emricasan, anti-CCL2 antibody (carlumab, or ABN912), Tasquinimod, ASLAN002, IMC-RON8, or GW3965. See for example Fleming et al.” Targeting Myeloid-Derived Suppressor Cells to Bypass Tumor-Induced Immunosuppression” Front Immunol. 2018; 9: 398.

In embodiments, the immune inhibitors are formulated in an appropriate aqueous solution for administration, wherein the third composition is administered as a separate composition from either of the first or second vaccine compositions. In embodiments, the immune inhibitor composition is administered at different times and days than the first and/or second vaccine composition.

Methods of Use

In embodiments provided herein are methods for inducing an antigen CD8+ T cell response via administration of a heterologous prime and boost dose of a vaccine composition. In certain embodiments, those methods comprise administering a first composition comprising a non-replicating viral vector encoding an antigen or immunogen containing one or more CD8+ T cell epitopes; and, administering a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen, wherein one of the first composition or the second composition is administered as a prime dose and the other one of the first composition or the second composition is administered as a boost dose, provided both the first and second compositions are administered.

In embodiments, the prime and boost dose are administered at least 7 days apart, at least 14 days apart, or longer. In embodiments, the prime dose and boost dose are administered about 7 days apart, about 14 days apart, about 20 days apart, about 25 days apart, about 30 days apart, about 35 days apart, about 40 days apart, about 45 days apart, about 50 days apart, about 55 days apart, about 60 days apart or about 65 days apart. Advantageously, the doses are administered about 40 days apart, about 41 days apart, about 42 days apart, about 43 days apart, about 44 days apart, about 45 days apart, about 46 days apart, about 47 days apart, about 48 days apart, about 49 days apart or about 50 days apart. In certain embodiments, the prime dose and boost dose are administered about 1 week apart, about 2 weeks apart, about 3 weeks apart, about 4 weeks apart, about 5 weeks apart, about 6 weeks apart, about 7 weeks apart, about 8 weeks apart, about 9 weeks apart, about 10 weeks apart, about 11 weeks apart or about 12 weeks apart. In certain other embodiments, the prime dose and boost dose are administered about 1 month apart, about 2 months apart, about 3 months apart, about 4 months apart, about 5 months apart, about 6 months apart, about 7 months apart, about 8 months apart, about 9 months apart, about 10 months apart, about 11 months apart, or about 12 months apart.

In certain embodiments, methods comprise administering a first composition comprising a non-replicating viral vector encoding an antigen or immunogen containing one or more CD8+ T cell epitopes; or, administering a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the antigen or immunogen, and administering separately a third composition comprising an immune modulator. In embodiments, the immune modulator is selected from an anti-immunosuppressor or an immunoactivator. The immunomodulator enhances the cell mediated immune response induced by the first and/or second composition.

In embodiments, the first or second vaccine composition is administered as a prime and boost dose administered at least 7 days apart, at least 14 days apart, or longer. In embodiments, the prime dose and boost dose are administered about 7 days apart, about 14 days apart, about 20 days apart, about 25 days apart, about 30 days apart, about 35 days apart, about 40 days apart, about 45 days apart, about 50 days apart, about 55 days apart, about 60 days apart or about 65 days apart. Advantageously, the doses are administered about 40 days apart, about 41 days apart, about 42 days apart, about 43 days apart, about 44 days apart, about 45 days apart, about 46 days apart, about 47 days apart, about 48 days apart, about 49 days apart or about 50 days apart. In certain embodiments, the prime dose and boost dose are administered about 1 week apart, about 2 weeks apart, about 3 weeks apart, about 4 weeks apart, about 5 weeks apart, about 6 weeks apart, about 7 weeks apart, about 8 weeks apart, about 9 weeks apart, about 10 weeks apart, about 11 weeks apart or about 12 weeks apart. In certain other embodiments, the prime dose and boost dose are administered about 1 month apart, about 2 months apart, about 3 months apart, about 4 months apart, about 5 months apart, about 6 months apart, about 7 months apart, about 8 months apart, about 9 months apart, about 10 months apart, about 11 months apart, or about 12 months apart.

In embodiments, the third composition, the immune modulator, is administered on the same days as the first or second vaccine compositions. In embodiments, the third composition is administered on multiple days, at least two days, at least three days, at least four days, at least five days or at least six days. In embodiments, the third composition is administered at least one time between a prime and boost dose of the first or second vaccine composition and at least one time after the boost dose of the first or second vaccine combination. In exemplary embodiments, the third composition is administered on day 4, 7, 11, 15, 18 and 22 after the prime dose administration of the first or second vaccine composition.

In embodiments, the vaccine combination is administered to a subject in need thereof. In embodiments, the subject in need thereof is a vertebrate such as a mammal, bird, reptile, amphibian, or fish. In certain embodiments, the subject is a human, a companion or domesticated or food-producing or feed-producing animal or livestock or game or racing or sport animal such as a cow, a dog, a cat, a goat, a sheep or a pig or a horse, or even fowl such as turkey, ducks or chicken. In certain embodiments, the vertebrate is a human.

As used herein, an immunologically effective amount is an amount or concentration of the compositions of the vaccine combination, that, when administered to a subject in need thereof, produces an immune response to the delivered antigen. In certain embodiments, the immunologically effective amount of the vaccine combination or vaccine compositions produces an antigen specific CD8+ T cell response.

The methods of the invention can be appropriately applied to prevent diseases as prophylactic vaccination or treat diseases as therapeutic vaccination. The vaccine combination of the present invention can be administered to a subject in need thereof either alone as a prime dose and boost dose or in combination with an immune inhibitor composition. Further the vaccine combination of the present invention can be administered to a subject in need thereof as a prime dose or a boost dose in combination with an immune inhibitor composition The immune modulator composition is a third distinct composition of the vaccine combination and can be administered at the same day and time as either the first vaccine composition and/or the second vaccine composition, or on a different day. In exemplary embodiments, the immune modulator composition is administered after the first vaccine composition.

In certain embodiments, the vaccine combination is administered to a subject in need thereof for use in treating or preventing cancer. In embodiments, the vaccine combination is used as a therapeutic or prophylactic for non-small-cell lung cancer, breast cancer, hepatic cancer, brain cancer, stomach cancer, pancreatic cancer, kidney cancer, ovarian cancer, myeloma cancer, acute myelogenous leukemia, chronic myelogenous leukemia, head and neck cancer, colorectal cancer, renal cancer, esophageal cancer, melanoma skin cancer and/or prostate cancer patients. Those cancer cells may express neoantigens or viral antigens, and the vaccine compositions will comprise appropriate polypeptides comprising one or more CD+8 T cell epitope depending on the biology of the particular cancer/tumor.

In certain embodiments, the vaccine combination is administered to a subject in need thereof for use in treating or preventing chronic infection. In embodiments, the vaccine combination is used as a therapeutic or prophylactic for either individuals with a chronic infection, or those at risk of exposure to pathogens that cause chronic infections. Such pathogens include, but are not limited to, HIV, hepatitis B and D viruses, herpesviruses, Epstein-Barr virus, cytomegalovirus and human T-lymphotropic virus type III.

The vaccine combination can be administered to a human or animal subject in vivo using a variety of known routes and techniques. For example, the compositions of the vaccine combination may be provided as an injectable solution, suspension or emulsion and administered via parenteral, subcutaneous, oral, epidermal, intradermal, intramuscular, intraarterial, intraperitoneal, intravenous injection using a conventional needle and syringe, or using a liquid jet injection system. The composition of the vaccine combination may be administered topically to skin or mucosal tissue, such as nasally, intratracheally, intestinally, sublingually, rectally or vaginally, or provided as a finely divided spray, such as a mist, suitable for respiratory or pulmonary administration. In certain embodiments, the vaccine compositions are administered intramuscularly.

The compositions of the vaccine combination can be administered to a subject in an amount that is compatible with the dosage composition that will be prophylactically and/or therapeutically effective. The administration of the composition of the invention may be for either “prophylactic” or “therapeutic” purpose. As used herein, the term “therapeutic” or “treatment” includes any one or more of the following: the prevention of infection; the treatment of chronic infection; the prevention of tumorigenesis/carcinogenesis; the reduction or elimination of symptoms; and/or the reduction or complete elimination of a tumor or cancer.

In embodiments, the vaccine combination comprises cancer antigens or viral antigens associated with cancer, wherein treatment may be prophylactic (prior to confirmed diagnosis of the cancer) or therapeutic (following diagnosis of the cancer). Therapeutic treatment may be given to Stage I, II, III, or IV cancers, pre- or post-surgical intervention. The treatment may be post-surgery maintenance treatment or a long-term treatment to improve progression free survival or overall survival and/or clearance of disease.

The choice of carrier, if required, is frequently a function of the route of delivery of the composition. Within this invention, compositions may be formulated for any suitable route and means of administration. Pharmaceutically acceptable carriers or diluents include those used in compositions suitable for oral, ocular, rectal, nasal, topical (including buccal and sublingual), vaginal or parenteral (including subcutaneous, intramuscular, intravenous, intradermal, transdermal) administration.

The compositions may be administered in any suitable form, for example as a liquid, solid or aerosol. For example, oral formulations may take the form of emulsions, syrups or solutions or tablets or capsules, which may be enterically coated to protect the active component from degradation in the stomach. Nasal formulations may be sprays, mists or solutions. Transdermal formulations can be adapted for their particular delivery system and may comprise patches. Formulations for injection may be solutions or suspensions in distilled water or another pharmaceutically acceptable solvent or suspending agent.

The appropriate dosage of the prophylactic or therapeutic vaccine to be administered to a patient will be determined in the clinic. Multiple doses, beyond the original prime and boost dose, may be required to achieve an immunological or clinical effect, which, if required, will be typically administered between 1 to 12 weeks apart. Where boosting of the immune response over longer periods is required, repeat doses 1 month to 5 years apart may be applied

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Preparation of a Vaccine Combination Comprising a Heterologous Prime and Boost Dose Combination: A First Composition (A Non-Replicating Viral Vector Composition); and, A Second Composition (Fluorocarbon-Linked Peptide Composition)

Non-Replicating Viral Vector Composition

AdGP70 was designed as a recombinant adenovirus serotype 5 vector rendered replication defective by the deletion of the E1 and E3 (4E1E3) and allowing the expression of the GP70 (the envelope (Env) protein a Murine leukemia virus—MuLV—GenBank accession number ABC94931.1) under the cytomegalovirus (CMV) promoter. First, a shuttle plasmid was obtained following cloning of a chemically synthesized, codon-optimized GP70 gene for expression in mammalian cells into the HindIII/XbaI restriction sites of pAdHighy shuttle vector (Altimmune Inc) at Genscript (Piscataway, N.J.). Recombination between the transgene-containing pAdHighy shuttle vector and the pAdEasy-1 adenovirus 5 backbone plasmid was performed in E. coli strain BJ5183 under kanamycin selection for genomic plasmid pAdGP70 generation. The pAdEasy-1 plasmid contains all Ad5 sequences except nucleotides 1-3,533 (encompassing the E1 genes) and nucleotides 28,130-30,820 (encompassing E3). A selected genomic plasmid pAdGP70 was re-transformed into E. coli strain DH10B cells under kanamycin selection through colony isolation. A selected pAdGP70 colony was amplified and purified. The AdGP70 recombinant adenovirus vector seed was generated by transfecting Pad linearized purified pAdGP70 genomic plasmid into adenovirus packaging cell lines HEK293. AdGP70 vector was propagated on HEK293 cells and purified by ultracentrifugation over a cesium chloride gradient. The purified Ad5 vectors were sterilized by 0.22-μm-pore-size filtration and stored −80° C. in A195 adenoviral storage buffer. AdGP70 titer (4×10¹¹ ifu/ml) was determined by using an Adeno-X rapid titer kit (Clontech, Mountain View, Calif.) on HEK293 cells. Before in vivo administration, AdGP70 was further diluted in PBS to 4×10¹⁰ ifu/ml.

SEQ ID NO. 1: Murine leukemia virus GP70 protein
sequence -Accession number ABC94931.1
MDTRRPRQGSDHTPDKTIMESTTLSKPFKNQVNPWGPLIVLLILGGVN
PVALGNSPHQVFNLSWEVTNGDRETVWAITGNHPLWTWWPDLTPDLCM
LAHGPFSPPPGPPCCSGSSDSTPGCSRDCEEPLTSYTPRCNTAWNRLK
LSKVTHAHNEGFYVCPGPHRPRWARSCGGPESFYCASWGCETTGRASW
KPSSSWDYITVSNNLTSDQATPVCKGNEWCNSLTIRFTSFGKQATSWV
TGHWWGLRLYVSGHDPGLIFGIRLKITDSGPRVPIGPNPVLSDRRPPS
RPRPTRSPPPSNSTPTETPLTLPEPPPAGVENRLLNLVKGAYQALNLT
SPDKTQECWLCLVSGPPYYEGVAVLGTYSNHTSAPANCSVASQHKLTL
SEVTGQGLCIGAVPKTHQVLCNTTQKTSDGSYYLAAPTGTTWACSTGL
TPCISTTILDLTTDYCVLVELWPRVTYHSPSYVYHQFERRAKYKREPV
SLTLALLLGGLTMGGIAAGVGTGTTALVATQQFQQLQAAMHDDLKEVE
KSITNLEKSLTSLSEVVLQNRRGLDLLFLKEGGLCAALKEECCFYADH
TGLVRDSMAKLRERLSQRQKLFESQQGWFEGLFNKSPWFTTLISTIMG
PLIILLLILLFGPCILNRLVQFIKDRISVVQALVLTQQYHQLKTIGDC
KSRE

The T-cell epitopes present in the antigen can be identified using various methods, including published epitopes, artificial neural network (ANN) or stabilized matrix method (SMM) (using the Immune Epitope Database and Analysis Resource website) or the SYFPEITHI website which is a database comprising more than 7000 peptide sequences known to bind class I and class II MHC molecules complied from published reports. T cell epitopes from GP70 were identified by predicting high affinity binding peptides for murine class I and class II MHC molecules (H-2Kd, H-2Dd, H-2Ld, IAd and IEd) using the artificial neural network (ANN) and stabilized matrix method (SMM) using a predicted IC50 cutoff of <50 nM and SYFPEITHI using a threshold of >20 (http://www.syfpeithi.de/). Provided in Table 1 are the T cell epitopes of the GP70 protein sequence identified using a combination of those methods.

TABLE 1
T cell epitopes of GP70

| | | | | | | BINDING | |
|---|---|---|---|---|---|---|---|
| | | | | MHC | | AFFINITY | |
| | N-TERM | C-TERM | | RESTRICTION | | (nM) OR | |
| SEQUENCE | POSITION | POSITION | METHOD | (CD4/CD8) | ALLELE | SCORE | |
| VNPWGPLIVLLILGG | 32 | 46 | SYFPEITHI | CD4 | IAd | 23 | |
| SEQ ID NO: 2 | | | | | | | |
| NPWGPLIVL | 33 | 41 | SYFPEITHI | CD8 | Ld | 22 | |
| SEQ ID NO: 3 | | | | | | | |
| PWGPLIVLLILGGVN | 34 | 48 | SYFPEITHI | CD4 | IAd | 20 | |
| SEQ ID NO: 4 | | | | | | | |
| GPLIVLLIL | 36 | 44 | SYFPEITHI | CD8 | Ld | 22 | |
| SEQ ID NO: 5 | | | | | | | |
| GPLIVLLILGGVNPV | 36 | 50 | SYFPEITHI | CD4 | IAd | 21 | |
| SEQ ID NO: 6 | | | | | | | |
| LGGVNPVALGNSPHQ | 44 | 58 | SYFPEITHI | CD4 | IAd | 27 | |
| SEQ ID NO: 7 | | | | | | | |
| NSPHQVFNL | 54 | 62 | SYFPEITHI | CD8 | Ld | 22 | |
| SEQ ID NO: 8 | | | | | | | |
| NLSWEVTNGDRETVW | 61 | 75 | SYFPEITHI | CD4 | IEd | 20 | |
| SEQ ID NO: 9 | | | | | | | |
| NGDRETVWAITGNHP | 68 | 82 | SYFPEITHI | CD4 | IAd | 24 | |
| SEQ ID NO: 10 | | | | | | | |
| TPDLCMLAL | 91 | 99 | SYFPEITHI | CD8 | Ld | 22 | |
| SEQ ID NO: 11 | | | | | | | |
| SYWGLEYRA | 103 | 111 | SYFPEITHI | CD8 | Kd | 22 | |
| SEQ ID NO: 12 | | | | | | | |
| SYTPRCNTA | 142 | 150 | ANN | CD8 | Kd | 25.22 nM | GP70- |
| SEQ ID NO: 13 | | | | | | | 142 |
| SYTPRCNTA | 142 | 150 | SYFPEITHI | CD8 | Kd | 22 | |
| SEQ ID NO: 13 | | | | | | | |
| RCNTAWNRLKLSKVT | 146 | 160 | SYFPEITHI | CD4 | IAd | 20 | |
| SEQ ID NO: 14 | | | | | | | |
| NTAWNRLKLSKVTHA | 148 | 162 | SYFPEITHI | CD4 | IAd | 21 | |
| SEQ ID NO: 15 | | | | | | | |
| NTAWNRLKLSKVTHA | 148 | 162 | SYFPEITHI | CD4 | IEd | 20 | |
| SEQ ID NO: 15 | | | | | | | |
| WNRLKLSKVTHAHNE | 151 | 165 | SYFPEITHI | CD4 | IAd | 20 | |
| SEQ ID NO: 16 | | | | | | | |
| NEGFYVCPGPHRPRW | 164 | 178 | SYFPEITHI | CD4 | IEd | 20 | |
| SEQ ID NO: 17 | | | | | | | |
| RSCGGPESF | 180 | 188 | SYFPEITHI | CD8 | Ld | 21 | |
| SEQ ID NO: 18 | | | | | | | |
| CETTGRASWKPSSSW | 196 | 210 | SYFPEITHI | CD4 | IAd | 22 | GP70- |
| SEQ ID NO: 19 | | | | | | | 196 |
| KPSSSWDYI | 204 | 212 | SYFPEITHI | CD8 | Ld | 20 | |
| SEQ ID NO: 20 | | | | | | | |
| DYITVSNNL | 210 | 218 | ANN | CD8 | Kd | 33.91 mM | |
| SEQ ID NO: 21 | | | | | | | |
| DYITVSNNL | 210 | 218 | SMM | CD8 | Kd | 46.55 nM | |
| SEQ ID NO: 21 | | | | | | | |
| DYITVSNNL | 210 | 218 | SYFPEITHI | CD8 | Kd | 24 | |
| SEQ ID NO: 21 | | | | | | | |
| ITVSNNLTSDQATPV | 212 | 226 | SYFPEITHI | CD4 | IAd | 22 | |
| SEQ ID NO: 22 | | | | | | | |
| TGHWWGLRLYVSGHD | 252 | 266 | SYFPEITHI | CD4 | IAd | 25 | |
| SEQ ID NO: 23 | | | | | | | |
| LYVSGHDPG | 260 | 268 | SYFPEITHI | CD8 | Kd | 22 | |
| SEQ ID NO: 24 | | | | | | | |
| VPIGPNPVL | 284 | 292 | ANN | CD8 | Ld | 38.04 nM | |
| SEQ ID NO: 25 | | | | | | | |
| VPIGPNPVL | 284 | 292 | SYFPEITHI | CD8 | Ld | 21 | |
| SEQ ID NO: 25 | | | | | | | |
| NPVLSDRRPPSRPRP | 289 | 303 | SYFPEITHI | CD4 | IEd | 20 | |
| SEQ ID NO: 26 | | | | | | | |
| NSTPTETPL | 311 | 319 | SYFPEITHI | CD8 | Ld | 21 | |
| SEQ ID NO: 27 | | | | | | | |
| TPTETPLTL | 313 | 321 | SYFPEITHI | CD8 | Ld | 21 | |
| SEQ ID NO: 28 | | | | | | | |
| PPAGVENRL | 325 | 333 | SYFPEITHI | CD8 | Ld | 21 | |
| SEQ ID NO: 29 | | | | | | | |
| KTQECWLCLVSGPPY | 351 | 366 | SYFPEITHI | CD4 | IAd | 23 | |
| SEQ ID NO: 30 | | | | | | | |
| PPYYEGVAVLGTYSN | 363 | 377 | SYFPEITHI | CD4 | IAd | 22 | |
| SEQ ID NO: 31 | | | | | | | |
| PYYEGVAVL | 364 | 372 | SYFPEITHI | CD8 | Kd | 21 | |
| SEQ ID NO: 32 | | | | | | | |
| YSNHTSAPANCSVAS | 375 | 389 | SYFPEITHI | CD4 | IAd | 29 | |
| SEQ ID NO: 33 | | | | | | | |
| NHTSAPANCSVASQH | 377 | 391 | SYFPEITHI | CD4 | IAd | 22 | |
| SEQ ID NO: 34 | | | | | | | |
| CSVASQHKL | 385 | 393 | SYFPEITHI | CD8 | Ld | 21 | |
| SEQ ID NO: 35 | | | | | | | |
| VASQHKLTLSEVTGQ | 387 | 401 | SYFPEITHI | CD4 | IAd | 20 | |
| SEQ ID NO: 36 | | | | | | | |
| SDGSYYLAAPTGTTW | 422 | 436 | SYFPEITHI | CD4 | IAd | 28 | |
| SEQ ID NO: 37 | | | | | | | |
| WACSTGLIPCISTTI | 437 | 451 | SYFPEITHI | CD4 | IAd | 22 | |
| SEQ ID NO: 38 | | | | | | | |
| TPCISTTIL | 444 | 452 | SYFPEITHI | CD8 | Ld | 22 | |
| SEQ ID NO: 39 | | | | | | | |
| SPSYVYHQF | 472 | 480 | ANN | CD8 | Ld | 30.26 nM | GP70- |
| SEQ ID NO: 40 | | | | | | | 472 |
| SPSYVYHQF | 472 | 480 | SYFPEITHI | CD8 | Ld | 22 | |
| SEQ ID NO: 40 | | | | | | | |
| SPSYVYHQFERRAKY | 472 | 486 | SYFPEITHI | CD4 | IEd | 26 | |
| SEQ ID NO: 41 | | | | | | | |
| YHQFERRAKYKREPV | 477 | 491 | SYFPEITHI | CD4 | IEd | 32 | |
| SEQ ID NO: 42 | | | | | | | |
| KYKREPVSL | 485 | 493 | SYFPEITHI | CD8 | Kd | 25 | |
| SEQ ID NO: 43 | | | | | | | |
| KYKREPVSLTLALLL | 485 | 499 | SYFPEITHI | CD4 | IAd | 24 | |
| SEQ ID NO: 44 | | | | | | | |
| EPVSLTLAL | 489 | 497 | SYFPEITHI | CD8 | Ld | 21 | |
| SEQ ID NO: 45 | | | | | | | |
| EPVSLTLALLLGGLT | 489 | 503 | SYFPEITHI | CD4 | IAd | 24 | |
| SEQ ID NO: 46 | | | | | | | |
| PVSLTLALLLGGLTM | 490 | 504 | SYFPEITHI | CD4 | IAd | 22 | |
| SEQ ID NO: 47 | | | | | | | |
| VSLTLALLLGGLIMG | 491 | 505 | SYFPEITHI | CD4 | IAd | 24 | |
| SEQ ID NO: 48 | | | | | | | |
| GLIMGGIAAGVGIGT | 501 | 515 | SYFPEITHI | CD4 | IAd | 26 | |
| SEQ ID NO: 49 | | | | | | | |
| GTGTTALVATQQFQQ | 512 | 526 | SYFPEITHI | CD4 | IAd | 22 | |
| SEQ ID NO: 50 | | | | | | | |
| TNLEKSLTS | 543 | 551 | SYFPEITHI | CD8 | Kd | 21 | |
| SEQ ID NO: 51 | | | | | | | |
| RRGLDLLFLKEGGLC | 560 | 574 | SYFPEITHI | CD4 | IAd | 22 | |
| SEQ ID NO: 52 | | | | | | | |
| LKEECCLYADHTGLV | 577 | 591 | SYFPEITHI | CD4 | IAd | 21 | |
| SEQ ID NO: 53 | | | | | | | |
| CCLYADHTGLVRDSM | 581 | 595 | SYFPEITHI | CD4 | IEd | 20 | |
| SEQ ID NO: 54 | | | | | | | |
| LYADHTGLV | 583 | 591 | SYFPEITHI | CD8 | Kd | 21 | |
| SEQ ID NO: 55 | | | | | | | |
| ADHTGLVRDSMAKLR | 585 | 599 | SYFPEITHI | CD4 | IAd | 24 | |
| SEQ ID NO: 56 | | | | | | | |
| RDSMAKLRERLSQRQ | 592 | 606 | SYFPEITHI | CD4 | IAd | 20 | |
| SEQ ID NO: 57 | | | | | | | |
| MAKLRERLSQRQKLF | 595 | 609 | SYFPEITHI | CD4 | IEd | 20 | |
| SEQ ID NO: 58 | | | | | | | |
| ERLSQRQKLFESQQG | 600 | 614 | SYFPEITHI | CD4 | IAd | 20 | |
| SEQ ID NO: 59 | | | | | | | |
| WFTTLISTI | 625 | 633 | ANN | CD8 | Kd | 6.17 nM | |
| SEQ ID NO: 60 | | | | | | | |
| WFTTLISTI | 625 | 633 | SMM | CD8 | Kd | 44.68 nM | |
| SEQ ID NO: 60 | | | | | | | |
| WFTTLISTI | 625 | 633 | SYFPEITHI | CD8 | Kd | 21 | |
| SEQ ID NO: 60 | | | | | | | |
| MGPLIILLLILLFGP | 634 | 648 | SYFPEITHI | CD4 | IAd | 22 | |
| SEQ ID NO: 61 | | | | | | | |
| PLIILLLILLFGPCI | 636 | 650 | SYFPEITHI | CD4 | IAd | 21 | |
| SEQ ID NO: 62 | | | | | | | |
| ILLFGPCILNRLVQF | 643 | 657 | SYFPEITHI | CD4 | IEd | 20 | |
| SEQ ID NO: 63 | | | | | | | |
| LVQFIKDRISVVQA | 654 | 667 | Casares N. | CD4 | H2d class | N.A. | |
| SEQ ID NO: 64 | | | et al * | | II | | |
| QFIKDRISVVQALVL | 656 | 670 | SYFPEITHI | CD4 | IAd | 24 | |
| SEQ ID NO: 65 | | | | | | | |
| KDRISVVQALVLTQQ | 659 | 673 | SYFPEITHI | CD4 | IAd | 27 | |
| SEQ ID NO: 66 | | | | | | | |
| ISVVQALVL | 662 | 670 | SYFPEITHI | CD8 | Ld | 20 | |
| SEQ ID NO: 67 | | | | | | | |

N Casares; J J Lasarte; A L de Cerio; P Sarobe: M Ruiz; I Melero; J Prieto; F Borrás-Cuesta. Immunization with a tumor-associated CTL epitope plus a tumor-related or unrelated Th1 helper peptide elicits protective CTL immunity. 2001. Eur J Immunol. 31: 1780-9.

Fluorocarbon-Linked Peptide Composition

The fluorocarbon-linked peptide composition (PepGP70) contains four fluorocarbon-modified peptides (GP70-142, GP70-196, GP70-472, GP70-CM) derived from the GP70, the envelope (Env) protein a Murine leukemia virus (MuLV). The sequences of GP-70-142, GP 70-196, GP70-472, GP70-CM are presented in Table 1 and Table 2. The four peptides were selected based on the prediction of high affinity binding peptides for murine class I and class II MHC molecules (H-2Kd, H-2Dd, H-2Ld, IAd and IEd) using the artificial neural network (ANN) and stabilized matrix method (SMM) using a predicted IC50 cutoff of <50 nM (www.iedb.org/) and SYFPEITHI using a threshold of >20 (www.syfpeithi.de/) and published information. GP70-142, GP70-196, GP70-472, GP70-CM were obtained by solid phase peptide synthesis (SPPS). All peptides were synthesized by American Peptide Company (Sunnyvale, Calif.) building the peptide on resin using the standard 9-fluorenyhnethoxycarbonyl (Fmoc) chemistry. The incorporation of the 2H,2H,3H,3H-Perfluoroundecanoic acid fluorocarbon chain (C₈F₁₇(CH₂)₂COOH) on the epsilon-chain of a selectively deprotected C- or N-terminal additional lysine to derive the fluorocarbon-modified peptides. After cleavage and removal of the side chain protecting groups, crude peptides were precipitated from cold ether and collected by filtration. Purity was assessed by RP-HPLC and was superior to 90% for all peptides. Freeze-dried fluorocarbon linked peptides were stored at −20° C. PepGP70 was obtained after solubilization of peptides GP-70-142, GP70-196, GP70-472, GP70-CM, blending, addition of mannitol/water solution, filtration using a 0.22 μm filter and freeze-drying to generate individual vials containing 1400 μg per peptide. Freeze-dried PepGP70 were stored at −20° C. Before in vivo administration, freeze-dried PepGP70 vials were reconstituted with 1.4 ml of 28 mM L-Histidine containing ODN1585 (TLR 9 agonist; Invivogen, Toulouse, France) leading to strength of 100 μg/ODN1585/ml and 1000 μg/peptide/ml.

TABLE 2
Four peptides of PepGP70 composition

| Name | Sequence |
|---|---|
| GP70-142 | SYTPRCNTAWNRLKLSKVTHAHNE(K)FA₁ |
| | (SEQ ID NO: 68) |
| GP70-196 | FA₁(K)ETTGRASWKPSSSWDYITVSNNLTSDQATPVKKK |
| | (SEQ ID NO: 69) |
| GP70-472 | SPSYVYHQFERRAKYKREPVSLTLALLLGGLTMGKKK(K) |
| | FA₁ (SEQ ID NO: 70) |
| GP70-CM | SPSYVYTHQFKKKLVQFIKDRISVVQA(K)FA₁ |
| | (SEQ ID NO: 71) |

FA₁ = C₈F₁₇(CH₂)₂-CO-

The above peptides derived from the GP70 protein sequence are underlined in the above full-length sequence of the protein (SEQ ID NO: 1) and T cell epitopes identified in Table 1. GP70-472 and GP70-CM contain the cytotoxic T lymphocyte (CTL) epitope SPSYVYHQF (SEQ ID NO: 72) (also referred to as AH1 in the dextramer). KKK is a linker and not present in the full-length GP70 protein sequence. GP70-CM is a chimeric peptide containing a CTL epitope (CD8+ epitope), a KKK linker and a T-helper lymphocyte (HTL) epitope.

Accordingly, provided herein is a vaccine combination for a heterologous prime boost dosing regimen wherein the combination comprises a first composition comprising a non-replicating viral vector encoding an antigen or immunogen containing one or more CD8+ T cell epitopes; and, a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more CD8+ T cell epitopes of the antigen or immunogen in the first composition; wherein either of the first or second composition is a prime composition or boost composition.

### Example 2: Preparation of a Vaccine Combination Comprising a T Cell Vaccine Composition and an Immune Modulator Composition Combination: A First Composition (a Non-Replicating Viral Vector Composition); or, a Second Composition (Fluorocarbon-Linked Peptide Composition); and, a Third Composition (an Immune Modulator Composition)

The non-replicating viral vector composition (the first composition) and the fluorocarbon-linked peptide composition (the second composition) were prepared as disclosed in Example 1.

Immune Modulator Composition

Immune modulator compositions can be any of those disclosed herein and provided in appropriate aqueous solution. In the instant example, anti-PD-1 was provided in PBS, and as disclosed below administered separately, and at a different time point, from either of the first or second vaccine compositions disclosed above.

Accordingly, provided herein is a vaccine combination for use in inducing CD8+ T cell response and reducing tumor size wherein the combination comprises a first composition comprising a non-replicating viral vector encoding an antigen or immunogen containing one or more CD8+ T cell epitopes; or, a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more CD8+ T cell epitopes of the antigen or immunogen; and, a third composition comprising an immune modulator selected from an anti-immunosuppressor or an immunoactivator. The first or second composition is administered separately from the third composition.

In certain embodiments provide herein are vaccine combinations comprising two to three compositions selected from: a) a first composition comprising a non-replicating viral vector encoding an antigen or immunogen containing one or more CD8+ T cell epitopes; b) a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen; and c) a third composition comprising an immune modulator selected from an anti-immunosuppressor or an immunoactivator, wherein when the first composition and second composition are selected, either of the first or second composition is a prime composition or boost composition.

### Example 3: Inducing an Antigen Specific CD8+ T Cell Response Using the Non-Replicating Viral Vector Composition and Fluorocarbon-Linked Peptide Composition; a Heterologous Vaccine Combination

Preparation of AdGP70 (non-viral vector composition) and PepGP70 (fluorocarbon-linked peptide composition) are disclosed in Example 1, wherein AdGP70 is a replication deficient Adenoviral vector expressing GP70 (SEQ ID NO: 1) and PepGP70 is a combination of four peptides (SEQ ID NOs: 68 to 71) individually attached to a fluorocarbon chain and present in micelles.

Comparison of a homologous prime/boost dosing and heterologous prime/boost dosing of the AdGP70 and PepGP70 compositions was performed, wherein immunogenicity of the AdGP70 and PepGP70 compositions following one or two administrations in BALB/c mice was compared to the heterologous prime/boost combinations of AdGP70 and PepGP70. Mice were immunized using the subcutaneous route with 14 days interval between administration of a prime dose and administration of a boost dose. Group 1 (n=8) received 50 μl of PepGP70 (50 ug/peptide) subcutaneously on day 0. Group 2 (n=8) received 50 μl of AdGP70 (2×10⁹ ifu) subcutaneously on day 0. Group 3 (n=8) received 50 μl of PepGP70 (50 ug/peptide) subcutaneously on day 0 and day 14. Group 4 (n=8) received 50 μl of PepGP70 (50 ug/peptide) subcutaneously on day 0 and day 14. Group 5 (n=8) received 50 μl of AdGP70 (2×10⁹ ifu) and PepGP70 (50 ug/peptide) subcutaneously on day 0 and day 14 respectively. Group 6 (n=8) received 50 μl of PepGP70 (50 ug/peptide) and AdGP70 (2×10⁹ ifu) subcutaneously on day 1 and day 14 respectively. 10 days after the final administration (measurement at day 24), spleen cells from each animal were isolated and processed through two types of immunoassays: (1) an in vitro IFN-γ ELISpot assay to evaluate the frequency of GP70-specific T cells in individual animals (FIGS. 1) and (2) a dextramer-staining assay by flow cytometry to measure the frequency of CD8+ T cells specific for H-2Ld-restricted epitope derived from gp70 (AH1 peptide, sequence SPSYVYHQF (SEQ ID NO: 72)). See FIGS. 1 and 2.

Clinical trials have demonstrated efficacy of T cell inducing vaccines against a number of diseases, and although many approaches for assessing protective T cell responses may be taken, the ELISpot assay has become established as the most suitable means of determining vaccine immunogenicity. Slota M. et al., ELISpot for measuring human immune responses to vaccines. Expert Rev Vaccines 2011; 10(3):299-306. For the IFN-γ ELISpot assay, ELISpot plates (MSIPS4510 Merck Millipore) were pre-coated with 100 μl/well of capture IFN-γ antibody diluted in PBS at 5 μg/ml (Mouse IFNg ELISPOT pair, BD, ref 551881) under aseptic conditions and incubated overnight at 4° C. The coating antibody was removed, and plates washed, then incubated 2 hr at room temperature with 200 μL/well of complete culture medium composed of RPMI Glutamax 1640 (GIBCO, ref 11548876) supplemented with 10% fetal bovine serum, (GIBCO, ref 10270-098), and 1% penicillin-streptomycin solution (GIBCO, ref 11548876). After removing the medium, spleen cell suspensions in complete culture medium were added at a concentration of 5×10⁵ cells per well to the pre-coated ELISpot plates in the presence of either 10 ug/ml of each individual peptide (GP-70-142, GP70-196, GP70-472, GP70-CM) or a four peptide mixture, concanavalin A as positive control (eBiosciences, ref 00-4978-03) in a volume of 200 μl per well. Media only was used as negative control. Each test condition was achieved in duplicate. Plates were incubated for 18 hours at 37° C., 5% CO2 in a humidified environment. After two washing steps with deionized water (DI) to completely remove the cells, plates were extensively washed with ELISpot wash buffer (1× Dulbecco's PBS, Gibco, Fisher Cat#11540486), 0.05% Tween®20 Fisher cat #10113103). Then, ELISpot plates were incubated 2 hrs at room temperature with 100 μl/well of anti-IFN-g detection antibody (Mouse IFNg ELISPOT pair, BD, ref 551881) diluted in PBS supplemented with 10% foetal bovine serum at 2 ug/ml. Detection antibody solution was discarded and plates washed with ELISpot wash buffer before to be incubated for 1 hr at room temperature with 100 μL/well diluted Streptavidin-HRP (BD™ ELISPOT Streptavidin-HRP, Cat. No. 557630). Streptavidin-HRP solution was removed and plates washed with ELISpot wash buffer. 100 μL of final substrate solution (AEC) were added to each well. Spot development was monitored from 15-20 min and substrate reaction stopped by washing wells with DI water. Plates were then air-dry overnight at room temperature in the dark. Spot enumeration was performed using an ELISPOT Analyzer: ImmunoSpot® ELISpot plate reader (CTL—Europe GmbH, Germany) The spot forming cells, SFC/well, were enumerated to quantify the number of cells producing IFN-γ in response to specific stimulus.

See FIG. 1, which shows the cumulative IFN-γ ELISpot responses to the four peptides expressed as the number of IFN-γ producing cells (spot forming cells, SFC) per million of spleen cells calculated for each group at day 24 post administration of the prime dose. Groups 5 and 6 are the heterologous prime boost groups showing a synergist effect as compared to the homologous prime boost groups.

For the dextramer assay, murine spleen cells were stained with H-2 Ld dextramer prepared with the AH1 peptide (CD8+ T cell epitope derived from GP70, sequence: SPSYVYHQF (SEQ ID NO: 73)) or an irrelevant control H-2 Ld dextramer prepared with the NP118-126 peptide (CD8+ T cell epitope derived from the nucleoprotein of LCMV, sequence: RPQASGVYM (SEQ ID NO: 74)), both labelled with Phycoerythrin (PE). Dextramer reagents consist of a dextran polymer backbone carrying an optimized number of MHC-peptide complexes and fluorochromes, offering the ability to detect antigen-specific T cells with the T cell receptor recognizing specifically the MHC/peptide complex carried by the dextran polymer. The cell staining process for flow cytometry analysis is described as follows. Individual spleen cells from group 1 to 6 animals (1×106 cells) were cultured in 200 μl of complete culture medium composed of RPMI Glutamax 1640 (GIBCO, ref 11548876) supplemented with 10% foetal bovine serum, (GIBCO, ref 10270-098), and 1% penicillin-streptomycin solution (GIBCO, ref 11548876) in a 96 well plates. Plates were incubated overnight at 37° C., 5% CO2 in a humidified environment. After incubation, spleen cells from individual mice were pooled by group and washed with staining buffer (DPBS1× supplemented with 5% of fetal bovine serum, 2 mM EDTA and 1% penicillin-streptomycin solution) by centrifugation for 6 min at 1300 rpm 4° C. Fc receptors were blocked by incubating the spleen cells for 10 minutes at 4° C. with 25 ul of cold staining buffer containing an anti-mouse CD16/32 antibody diluted to 1:200. Then, cells were stained with either the relevant AH-1/H-2 Ld dextramer (Immudex, ref JG3294-OPT) or irrelevant NP118-126/H-2 Ld dextramer (Immudex, ref JG2750-OPT). 25 ul of 1:2.5 appropriate pre-diluted dextramer in staining buffer was added to Fc receptor blocked cells following by incubation for 30 minutes at 4° C. After incubation, a 2× antibody cocktail containing anti-mouse CD4 Pe-Cy7 (Ozyme, ref BLE100422), anti-mouse CD8a BV510 (Ozyme, ref BLE100752), anti-mouse CD44 BV650 (Ozyme, ref BLE103049), Anti-mouse CD62L BV711 (Ozyme, ref BLE104445), anti-mouse CD45 APC (Ozyme, ref BLE103112), anti-mouse PD1 PERCP-Cy5.5 (Ozyme, ref BLE109119), anti-mouse CD69 APC-eFluor780 (eBioscience, ref 47-0691-80) and Viability Dye eFluor™ 520 (eBioscience, ref 65-0867-14) was prepared in staining buffer. 50 ul of antibody cocktail was added on dextramer stained cells following by incubation 20 minutes at 4° C. After the staining steps, cells were washed twice with staining buffer by centrifugation for 6 min at 1300 rpm 4° C. and resuspended in 200 ul of staining buffer before flow cytometry acquisition and analysis. Events were gated on alive, CD45+CD8 cells. FIG. 2 shows the percentage of CD8+ T cells positive for dextramer staining.

See FIG. 2, which shows the percentage of CD8+ T cells positive for dextramer staining at day 24 post administration of the prime dose. Groups 5 and 6 are the heterologous prime boost groups showing a synergist effect as compared to the homologous prime boost groups.

The results from the ELISpot assay demonstrate an increase in the number of IFN-gamma producing spleen cells as compared to a single administration versus two administrations of PepGP70 or AdGP70. Surprisingly, a heterologous prime-boost dose administration (AdGP70 followed by PepGP70 or the opposite) induced a stronger peripheral immune response as compared to a homologous prime-boost dose administration with AgGP70 or PepGP70. See FIGS. 1 and 2. The data reveal an unexpected synergy between the two-different types of immunogens in their ability to promote more robust T cell responses including CD8+ T cell responses when combined in a heterologous prime-boost dose administration. The results also indicate that a heterologous prime-boost regimen using the PepGP70 first and followed by the AdGP70 administration represent a more favorable condition for the induction of antigen-CD8+ T cells as revealed by the dextramer immunoassay. See FIG. 2.

Accordingly, provided herein is a method of inducing an immune response in a subject in need thereof using a heterologous dose regimen, wherein the method comprises administering a first composition comprising a non-replicating viral vector encoding an antigen or immunogen comprising one or more CD8+ T cell epitopes; and, administering a second composition comprising micelles containing fluorocarbon-linked peptides, wherein each peptide linked to the fluorocarbon is: i) 15 to 75 amino acid residues long; ii) from the antigen or immunogen of the first composition; and, iii) comprises one or more CD8+ T cell epitopes within the antigen or immunogen of the viral vector, wherein an antigen specific CD8+ T cells response is induced. The first composition or the second composition is administered as a prime dose and one of the first composition or the second composition is administered as a boost dose, provided both the first and second compositions are administered.

### Example 4: Use of the Non-Replicating Viral Vector Composition and Fluorocarbon-Linked Peptide Composition in a Heterologous Dosing Regimen

The synergy between AdGP70 and PepGP70 is examined by assessing the antitumor activity in BALB/c mice challenged with CT26 tumor cells. 6-8-week-old female BALB/c mice are used for the experiment. On day 0, 2×104 CT26 cell in 100 ul PBS are subcutaneously injected in the flank of mice. The vaccine composition of AdGP70 and. PepGP70 are prepared according to Example 1 wherein formulated vaccines are prepared as 50 ul of injectable solutions. The compositions are subcutaneously administrated according to a prime/boost schedule with 14 days between administration of the prime dose and boost dose.

Group 1 received 50 μl of PepGP70 (50 μg/peptide) subcutaneously on day 1 and day 14 respectively. Group 2 received 50 μl of AdGP70 (2×10⁹ ifu) subcutaneously on day 7 and day 14 respectively. Group 3 received only vaccine 50 μl of AdGP70 (2×10⁹ ifu) and PepGP70 (50 μg/peptide) subcutaneously on day 7 and day 14 respectively. Group 4 received 50 μl of PepGP70 (50 ug/peptide) and 50 μl of AdGP70 (2×10⁹ ifu) subcutaneously on day 1 and day 14 respectively. Group 5 received no treatment.

Both vaccines PepGP70 and AdGP70 tested individually or as prime-boost combinations to promote an anti-tumor response. Vaccine regimen consisting of a prime/boost combination promotes better antitumor responses compared to PepGP70 and AdGP70 tested individually.

### Example 5: Use of the Non-Replicating Viral Vector Composition and Fluorocarbon-Linked Peptide Composition in a Heterologous Dosing Regimen in Combination with an Immune Checkpoint Inhibitor (Anti-PD1)

Preparation of AdGP70 (non-viral vector composition) and PepGP70 (fluorocarbon-linked peptide composition) are disclosed in Example 1, wherein AdGP70 is a replication deficient Adenoviral vector expressing GP70 (SEQ ID NO: 1) and PepGP70 is a combination of four peptides (SEQ ID NOs: 68 to 71) individually attached to a fluorocarbon chain and present in micelles; and, preparation of the immune checkpoint inhibitor composition is disclosed in Example 2.

The synergy between AdGP70 and PepGP70 in combination with anti-PD1 treatment was examined by assessing the vaccine induced immune response in BALB/c mice challenged with CT26 tumor cells. 6-8-week-old female BALB/c mice (n=12 per group) were used for the experiment. Animals were anesthetized with a mixture of ketamine-domitor (0.7/80 mg/kg) to allow the animals to be shaved at the site of tumor cell inoculation and awaking induced by an injection antisedan (2 mg/kg). On day 0, 2×10⁴ CT26 cell in 100 ul PBS were subcutaneously injected in the flank of mice.

The vaccine composition of AdGP70 and PepGP70 were prepared according to Example 1 wherein formulated vaccines were prepared as 50 ul of injectable solutions. The compositions were subcutaneously administrated according to a prime/boost schedule with 7 days between administration of the prime dose and boost dose. In this tumor experimental model, a boost dose is administered after 7 days, instead of the typical 14 days, to ensure a robust immune response is mounted to detect the effect of the immune response against the tumor. Six injections of the immuno-checkpoint inhibitor composition, anti-PD1 (anti-CD279—clone RMP1-14, InVivoPlus, Euromedex, ref BP0146-100 mg; GoInVivo, Ozyme, ref BLE114115) were administrated by intra-peritoneal route.

Group 1 (n=12) received 50 μl of PepGP70 (50 μg/peptide) subcutaneously on day 7 and day 14 respectively and 200 μg of anti-mouse PD1 in 100 μl intraperitoneally on days 7, 11, 14, 18, 22 and 25. Group 2 (n=12) received 50 μl of AdGP70 (2×10⁹ ifu) subcutaneously on day 7 and day 14 respectively and 200 μg of anti-mouse PD1 in 100 μl intraperitoneally on days 7, 11, 14, 18, 22 and 25. Group 3 (n=12) received only vaccine 50 μl of AdGP70 (2×10⁹ ifu) and PepGP70 (50 μg/peptide) subcutaneously on day 7 and day 14 respectively and 200 μg of anti-mouse PD1 in 100 μl intraperitoneally on days 7, 11, 14, 18, 22 and 25. Group 4 (n=12) received 50 μl of PepGP70 (50 ug/peptide) and 50 μl of AdGP70 (2×10⁹ ifu) subcutaneously on day 7 and day 14 respectively and 200 μg of anti-mouse PD1 in 100 μl intraperitoneally on days 7, 11, 14, 18, 22 and 25. Group 5 received 200 μg of anti-mouse PD1 in 100 μl intraperitoneally on days 7, 11, 14, 18, 22 and 25. Group 6 (n=12) received no treatment.

For the dextramer assay, PBMCs collected at day 6 (D6) or day 20 (D20) were stained with H-2 Ld dextramer prepared with the AH1 peptide (CD8+ T cell epitope derived from GP70, sequence: SPSYVYHQF (SEQ ID NO: 73)) labelled with Phycoerythrin (PE). The cell staining process for flow cytometry analysis is described as follows. 200 μl of whole blood samples from group 1 to 6 animals were collected via retro orbital bleeding technique into EDTA coated tube and invert several times to prevent clotting. Red blood cells were lysed using multi-species RBC lysis buffer according manufacturer recommendations (eBiosciences, ref 00-4300-54). Individual PBMC samples from group 1 to 6 animals were cultured in 200 μl of complete culture medium composed of RPMI Glutamax 1640 (GIBCO, ref 11548876) supplemented with 10% foetal bovine serum, (GIBCO, ref 10270-098), and 1% penicillin-streptomycin solution (GIBCO, ref 11548876) in a 96 well plates. Plates were incubated overnight at 37° C., 5% CO2 in a humidified environment. After completion of red blood cell lysis, incubation, PBMCs from individual mice were washed with staining buffer (DPBS1X supplemented with 5% of fetal bovine serum, 2 mM EDTA and 1% penicillin-streptomycin solution) by centrifugation for 6 min at 1300 rpm at room temperature. Fc receptors were blocked by incubating the spleen cells for 10 minutes at 4° C. with 25 ul of cold staining buffer containing an anti-mouse CD16/32 antibody diluted to 1:200. Cells were then stained with either the relevant AH-1/H-2 Ld dextramer (Immudex, ref JG3294-OPT) or irrelevant NP118-126/H-2 Ld dextramer (Immudex, ref JG2750-OPT). 25 μl of 1:2.5 appropriate pre-diluted dextramer in staining buffer was added to Fc receptor blocked cells following by incubation for 30 minutes at 4° C. After incubation, a 2× antibody cocktail containing anti-mouse CD4 Pe-Cy7 (Ozyme, ref BLE100422), anti-mouse CD8a BV510 (Ozyme, ref BLE100752), anti-mouse CD44 VioBlue anti mouse (Miltenyi, ref 130-102-443), anti-mouse PD1 PERCP-Cy5.5 (Ozyme, ref BLE109119), CD25 PEeFluor 610 (eBioscience, ref 61-0251-82), CD11b Alexa 700 (eBioscience, ref 56-0112-82) and Viability Dye eFluor™ 520 (eBioscience, ref 65-0867-14) was prepared in staining buffer. 50 μl of antibody cocktail was added on dextramer stained cells following by incubation 20 minutes at 4° C. After the staining steps, cells were washed twice with staining buffer by centrifugation for 6 min at 1300 rpm 4° C. Surface stained cells were resuspended in 200 μl of Fixation/Permeabilization working solution prepared according manufacturer recommendations (Fixation/Permeabilization and Permeabilization buffers set kit, eBioscience, ref 00-5523-00), and incubated 25 minutes at 4° C. Cells were then washed with 1× Permeabilization buffer by centrifugation for 6 min at 1300 rpm 4° C. before to be stained with an anti-mouse Perforin APC (eBioscience, ref 17-9392-80). 50 μl of pre-diluted Perforin APC was added on dextramer stained cells following by incubation 20 minutes at 4° C. Cells were washed twice with 1× Permeabilization buffer by centrifugation for 6 min at 1300 rpm 4° C. then resuspended in staining buffer before flow cytometry acquisition and analysis. Events were gated on alive, Dextramer+CD8+ cells. FIG. 3 shows the percentage of CD8+ T cells positive for dextramer staining expressed as median value for each group at day 6 (D6) and day 20 (D20).

Both vaccine compositions PepGP70 and AdGP70 tested individually or as prime-boost combinations synergize with the anti-PD1 treatment in their ability to promote an anti-tumor immune response as presented in FIG. 3, wherein antigen-specific CD8+ T cells were measured at day 6 (D6) and day 20 (D20). The vaccine combination consisting of a prime with PepGP70 and a boost with AdGP70 with the anti-PD1 treatment promotes a much higher response compared to other vaccine regimens. All vaccine dosing regimen (Groups 1 to 4) synergize with anti-PD1, but the dosing regimen with PepGP70 as the prime dose and AdGP70 as the boost dose (Group 4) demonstrated synergy compared to homologous dosing regimen vaccines (Groups 1 and 2).

### Example 6: Use of the Fluorocarbon-Linked Peptide Composition in Homologous Dosing Regimen to Induce an Antigen Specific CD4+ and CD8+ T Cell Response and Antitumor Activity

The fluorocarbon-linked peptide (FP-OVA) composition was prepared according to the disclosure in Example 1, except an OVA peptide was used in place of GP70 peptides. FP-OVA is composed of an ovalbumin-derived peptide (sequence ISQAVHAAHAEINEAGRESIINFEKLTEWT (SEQ ID NO:75)) containing the CD4+ T cell epitope (Ova 323-339, sequence ISQAVHAAHAEINEAGR (SEQ ID NO:76)) and the CD8+ T cell epitope (amino-acid position 257-264, sequence SIINFEKL (SEQ ID NO:77)).

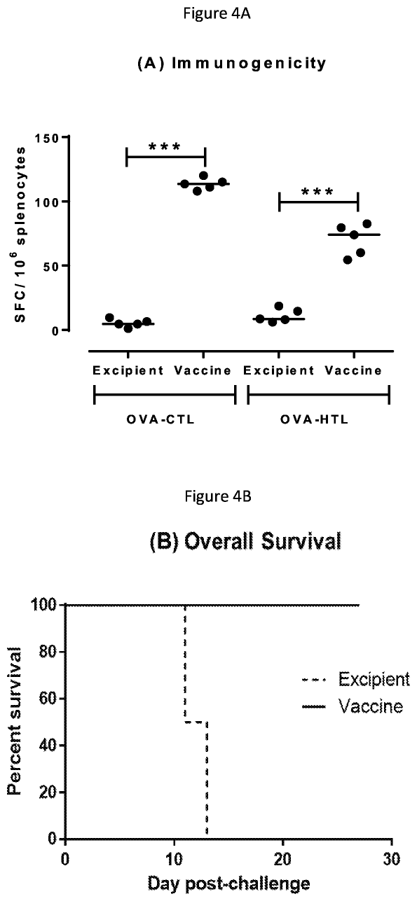

In this experiment, female C57BL/6 mice (n=15/group) were vaccinated subcutaneously at day 0 and day 14 with 200 ug of FP-OVA vaccine in 100 ul (group 1) or 100 ul of excipient solution (group 2). At day 24, 5 mice were sacrificed in each group to measure the immune response by mean of an ELISpot assay Immune response to the vaccine composition was measured as follows: spleens were harvested and cells were stimulated with 1 ng/ml OVA-CTL epitope (Ova 257-264, sequence SIINFEKL(SEQ ID NO:77)(CD8+ T cell epitope)) or 10 μg/mL OVA-HTL (Ova 323-339, sequence ISQAVHAAHAEINEAGR (SEQ ID NO:76) (CD4+ T cell epitopes)) in an IFNγ ELISpot assay. The number of IFNγ spot forming cells (SFC) were counted. FIG. 4A shows the number of IFNγ spot forming cells (SFC)/10⁶ splenocytes produced in response to OVA-CTL or OVA-HTL, for individual mice, following subtraction of the control well values (the line represents the median response value). Data were defined as parametric using a Kolmogorov-Smirnov normality test and statistical analyses were performed using a Student's t test, where P<0.001=***.

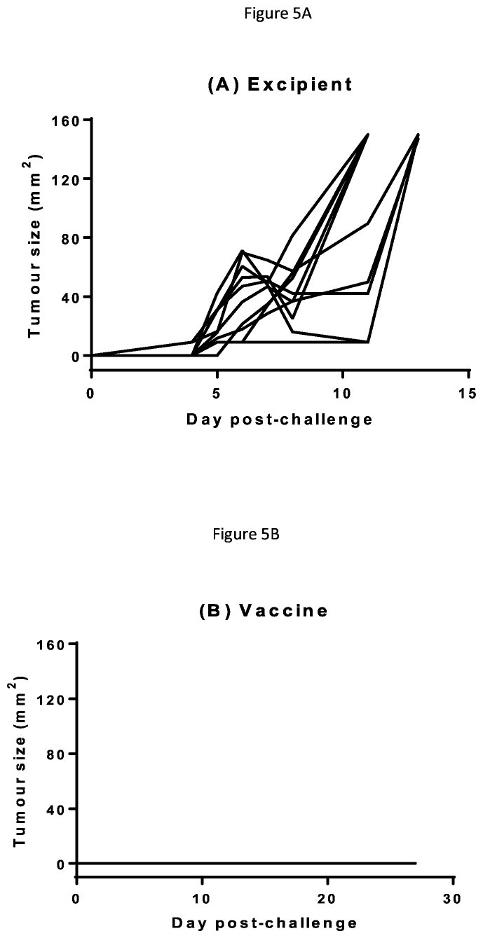

At day 24, the 10 remaining mice in each group received a subcutaneous injection of 2×10⁶ E.G7-OVA cells; mouse thymoma EL4 cells stably transfected with the complementary DNA of chicken ovalbumin (OVA) and thus express OVA epitopes as a unique antigen. Challenged mice were culled when tumor sizes reached 150 mm2 FIG. 4B shows the overall survival measured over time in the two different groups. FIG. 5 shows the tumor growth measured over time in the excipient group (FIG. 5A) and the vaccine group (FIG. 5B). FIGS. 4 and 5 demonstrate the effectiveness of fluorocarbon-linked peptides, wherein the peptides are unique cancer antigens, as a prophylactic against cancer.

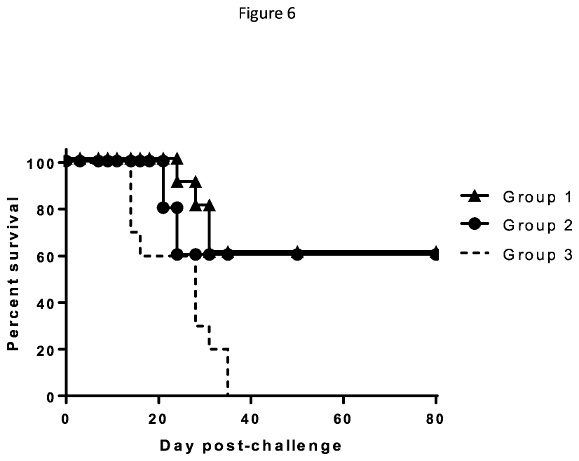

In a second experiment, female C57BL/6 mice (n=10/group) received subcutaneous injection of a subcutaneous injection of 2×10⁶ E.G7-OVA cells in one flank at day 0 and a second administration of 2×10⁶ E.G7-OVA cells in surviving animal on the opposite flank at day 50. Group 1 then received 200 ug of FP-OVA vaccine in 100 ul subcutaneously at day 1 and day 8. Group 2 received 200 ug of FP-OVA vaccine in 100 ul subcutaneously at day 3 (corresponding to the day of detection of a palpable tumor in at least one animal) and day 10. Group 3 received 100 ul of excipient subcutaneously at day 1 and day 8. Challenged mice were culled when tumor sizes reached 150 mm2 FIG. 6 shows the overall survival measured overtime in the different groups, wherein each of group 1 and 2 had a survival rate of at least 60% demonstrating the effectiveness of fluorocarbon-linked peptides, wherein the peptides are unique cancer antigens, as a therapeutic for cancer.

### Example 7: Use of the Non-Replicating Viral Vector Composition or Fluorocarbon-Linked Peptide Composition in Combination with an Immune Checkpoint Inhibitor (Anti-PD1)

Preparation of AdGP70 (non-viral vector composition) and PepGP70 (fluorocarbon-linked peptide composition) are disclosed in Example 1, wherein AdGP70 is a replication deficient Adenoviral vector expressing GP70 (SEQ ID NO: 1) and PepGP70 is a combination of four peptides (SEQ ID NOs: 68 to 71) individually attached to a fluorocarbon chain and present in micelles; and, preparation of the immune checkpoint inhibitor composition is disclosed in Example 2.

The synergy between AdGP70 or PepGP70 with anti-PD1 treatment was examined by assessing their respective antitumor activities against the CT26 colon carcinoma tumor in BALB/c mice. 6-8 weeks female BALB/c mice (n=12 per group) were used for the experiment. Animals were anesthetized with a mixture of ketamine-domitor (0.7/80 mg/kg) to allow the animals to be shaved at the site of tumor cell inoculation and awaking induced by an injection antisedan (2 mg 1 kg). On day 0, 2×10\hat{ }4 CT26 cell in 100 ul PBS were injected in the flank of mice. Tumor palpation was performed five times a week to determine the date of first positive tumor detection. Once a solid tumor was detected, its size was measured approximately three times a week. Tumor size measurements was performed with a caliper on two dimensions and volume determined according to the formula: L*1\hat{ }2/2; (L=longer axis and 1=shorter axis) Animals were sacrificed according to the following humane end-points: tumor volume≥2000 mm³, presence of necrotic or ulcerated tumor, impaired mobility including transient prostration or hunched posture, interference with a vital physiological function including respiration, significant abdominal distension or >20% weight loss in a week time.

Formulated vaccine compositions were prepared according to Example 1 and 2 as 50 ul of injectable solutions and were subcutaneously administrated according to a prime/boost schedule with 14 days between a prime dose and boost dose administration. Six injections of immuno-checkpoint, anti-PD1 (anti-CD279-clone RMP1-14, InVivoPlus, Euromedex, ref BP0146-100 mg; GoInVivo, Ozyme, ref BLE114115) were administrated by intra-peritoneal route, every 3 or 4 days after tumor initiation.

Group 1 (n=15) received only vaccine composition preparations, 50 μl of PepGP70 (50 ug/peptide) on day 1 and day 15 respectively. Group 2 (n=14) received 50 μl of PepGP70 (50 ug/peptide) on day 1 and day 15 respectively and 200 μg of anti-mouse PD1 in 100 ul delivery dose (anti-CD279-clone RMP1-14) on days 4, 7, 11, 15, 18 and 22. Group 3 (n=14) received only vaccine preparations, 50 μl of AdGP70 (2×10⁹ ifu) on day 0 and day 14 respectively. Group 4 (n=15) received 50 μl of AdGP70 (2×10⁹ ifu) on day 0 and day 14 respectively and 200 μg of anti-mouse PD1 in 100 ul delivery dose (anti-CD279-clone RMP1-14) on days 4, 7, 11, 15, 18 and 22. Group 5 (n=15) received 50 ul of vaccine preparation containing only TLR9 agonist on day 1 and day 15 respectively and 200 μg of anti-mouse PD1 in 100 ul delivery dose (anti-CD279-clone RMP1-14) on days 4, 7, 11, 15, 18 and 22. Group 6 (n=12) received no treatment. Results are presented in FIG. 7 (tumor volume in individual animal) and FIGS. 8A and 8B (overall survival for each group).

Both compositions PepGP70 and AgGP70 promote an anti-tumor activity with the delayed tumor growth and improved overall survival as presented in FIG. 7 and FIGS. 8A and 8B, respectively. In addition, both vaccines compositions benefit from the combination treatment with anti-PD1 showing a superior overall survival and delayed tumor growth profile compared to animal that have received only the vaccine compositions or the anti-PD1 treatment.

### Example 8: Use of the Non-Replicating Viral Vector Composition or Fluorocarbon-Linked Peptide Composition in Combination with Myeloid-Derived Suppressor Cell (MDSC) Inhibitor Composition

AdGP70 or PepGP70 in combination with an MDSC inhibitor is examined by assessing the anti-tumor activity in BALB/c mice challenged with CT26 tumor cells. 6-8-week-old female BALB/c mice are used for the experiment. On day 0, 2×10⁴ CT26 cell in 100 μl PBS are subcutaneously injected in the flank of mice. The vaccine composition of AdGP70 and PepGP70 are prepared according to Example 1 wherein formulated vaccines are prepared as 50 ul of injectable solutions. The compositions are subcutaneously administrated according to a prime/boost schedule with 14 days between administration of the mime dose and boost dose with individual animals receiving either a dose of AdGP70 or a dose of PepGP70.

Group 1 is administered 50 μl of PepGP70 (50 μg/peptide) subcutaneously on day 1 and day 14 and the MDSC inhibitor administered daily intraperitoneally. Group 2 is administered 50 μl of AdGP70 (2×10⁹ ifu) subcutaneously on day 1 and day 14. Group 3 is administered the MDSC inhibitor daily intraperitoneally. Group 4 is administered no treatment.

We claim:

1. A vaccine combination comprising two to three compositions selected from:
a) a first composition comprising a non-replicating viral vector encoding an antigen or immunogen containing one or more CD8+ T cell epitopes;
b) a second composition comprising micelles containing fluorocarbon-linked peptides, wherein each peptide linked to the fluorocarbon is: i) 15 to 75 amino acid residues long; ii) from the antigen or immunogen of the first composition; and, iii) contains one or more of the CD8+ T cell epitopes of the first composition from the antigen or immunogen; and
c) a third composition comprising an immune modulator selected from an anti-immunosuppressor or an immunoactivator,
wherein when the first composition and second composition are selected, either of the first or second composition is a prime composition or boost composition.

2. The vaccine combination of claim 1, wherein the anti-immunosuppressor targets PD1, PDL1, PDL2, CD28, CD80, CD86, CTLA4, B7RP1, ICOS, B7RPI, B7-H3, B7-H4, BTLA, HVEM, KIR, TCR, LAGS, CD 137, CD137L, OX40, OX40L, CD27, CD70, CD40, CD40L, TIM3, GAL1, GAL3, GAL9, ADORA, CD276, VTCN1, IDO1, KIR3DL1, HAVCR2, VISTA, CD244, ADAM17, COX2, PGE-2, iNOS2, PDE5, c-kit, ARG1, PI3K, CSF-1R, Caspase-8, CCL2, RON, ROS, or S100A8/A9.

3. The vaccine combination of claim 2, wherein the anti-immunosuppressor targets PD1 or PDL1.

4. The vaccine combination of claim 2, wherein the anti-immunosuppressor is an anti-PD1 or anti-PDL1 antibody.

5. The vaccine combination of claim 1, wherein the immunoactivator targets Toll-like receptor (TLR) 3, TLR4, TLR5, TLR7, TLR8, TLR9, NOD1, NOD2, STING, cGAS, IFR3, IL-2 receptor, IL12 receptor or IFN-alpha receptor.

6. The vaccine combination of claim 1, wherein the non-replicating viral vector is an adenovirus vector, an alphavirus vector, a herpesvirus vector, a measles virus vector, a poxviruses vector, or a vesicular stomatitis virus vector.

7. The vaccine combination of claim 6, wherein the non-replicating viral vector is an E1 and E3 deleted adenovirus vector.

8. The vaccine combination of claim 1, wherein the first, second or third composition is formulated for parenteral, oral, ocular, rectal, nasal, transdermal, topical or vaginal administration.

9. The vaccine combination of claim 1, wherein the antigen or immunogen is from a pathogen.

10. The vaccine combination of claim 9, wherein the pathogen is a virus, fungus, parasite, or bacteria.

11. The vaccine combination of claim 10, wherein the virus is EBV, HPV, HTLV-1, MCPvV, KSHV, HERV, HCV or HBV.

12. The vaccine combination of claim 1, wherein the antigen or immunogen is a cancer antigen.

13. The vaccine combination of claim 1, wherein the first composition is a prime composition and the second composition is a boost composition.

14. The vaccine combination of claim 1, wherein the second composition is a prime composition and the first composition is a boost composition.

15. The vaccine combination of claim 1, wherein the fluorocarbon portion of the fluorocarbon-linked peptide is a fluorocarbon chain from 3 to 30 carbon atoms.

16. The vaccine combination of claim 15, wherein one or more fluorine moieties of the fluorocarbon chain is substituted with chlorine, bromine, iodine, hydrogen or a methyl group.

17. The vaccine combination of claim 1, wherein the fluorocarbon-linked peptide is of structure CₘFₙ—C_{y}Hₓ-(Sp)-R, wherein m=3 to 30, n<=2m+1, y=0 to 15, x<=2y, (m+y)=3-30, Sp is an optional chemical spacer moiety and R is the peptide.

18. The vaccine combination of claim 1, wherein the fluorocarbon linked peptide is according to structure 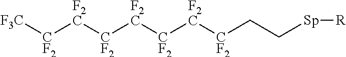
where Sp is an optional chemical spacer moiety and R is the peptide.

19. The vaccine combination of claim 1, wherein the fluorocarbon linked peptide of the second composition comprises at least one MHC class II binding epitope and at least one MHC class I binding epitope.

20. The vaccine combination of claim 1, wherein each of the first, second or third composition independently further comprise one or more pharmaceutically acceptable carriers, excipients, diluents or adjuvants.

21. The vaccine combination of claim 20, wherein the adjuvant is an agonist of TLR2, TLR3, TLR7, TLR8 or TLR9, STING, cGAS, IFR3, NOD1 or NOD2.

22. The vaccine combination of claim 1, wherein the first, second or third composition is in a form of a liquid, emulsion, solid, aerosol, mist or gas.

23. The vaccine combination of claim 1, wherein the first composition and second composition are selected.

24. The vaccine combination of claim 1, wherein one of the first composition or second composition is selected, and the third composition is selected.

25. A method of inducing an immune response in a subject in need thereof, comprising:
a) administering a first composition comprising a non-replicating viral vector encoding an antigen or immunogen containing one or more CD8+ T cell epitopes; and,
b) administering a second composition comprising micelles containing fluorocarbon-linked peptides, wherein each peptide linked to the fluorocarbon is: i) 15 to 75 amino acid residues long; ii) from the antigen or immunogen of the first composition; and,
iii) contains one or more of the CD8+ T cell epitopes of the first composition from the antigen or immunogen,
wherein one of the first composition or the second composition is administered as a prime dose and one of the first composition or the second composition is administered as a boost dose, provided both the first and second compositions are administered, whereby an antigen specific CD8+ T cells response is induced.

26. The method of claim 25, wherein the prime dose and boost dose are administered at least 14 days apart.

27. The method of claim 25, wherein the second composition is the prime dose and the first composition is the boost dose.

28. The method of claim 25, wherein the first composition is the prime dose and the second composition is the boost dose

29. The method of claim 25, wherein the subject is mammal, bird, reptile, amphibian, or fish.

30. The method of claim 2, wherein the subject is a human, a cow, a dog, a cat, a goat, a sheep, a pig, a horse, a turkey, duck or chicken.

31. The method of claim 25, further comprising administering a third composition comprising an immune modulator selected from an anti-immunosuppressor or an immunoactivator.

32. The method of claim 31, wherein the anti-immunosuppressor targets PD1, PDL1, PDL2, CD28, CD80, CD86, CTLA4, B7RP1, ICOS, B7RPI, B7-H3, B7-H4, BTLA, HVEM, KIR, TCR, LAG3, CD 137, CD137L, OX40, OX40L, CD27, CD70, CD40, CD40L, TIM3, GAL1, GALS, GALS, ADORA, CD276, VTCN1, IDO1, KIR3DL1, HAVCR2, VISTA, CD244, ADAM17, COX2, PGE-2, iNOS2, PDE5, c-kit, ARG1, PI3K, CSF-1R, Caspase-8, CCL2, RON, ROS, or S100A8/A9.

33. The method of claim 31, wherein the anti-immunosuppressor targets PD1 or PDL1.

34. The method of claim 31, wherein the anti-immunosuppressor is an anti-PD1 or anti-PDL1 antibody.

35. The method of claim 31, wherein the immunoactivator targets Toll-like receptor (TLR) 3, TLR4, TLR5, TLR7, TLR8, TLR9, NOD1, NOD2, STING, cGAS, IFR3, IL-2 receptor, IL12 receptor or IFN-alpha receptor.

36. The method of claim 31, wherein the step of administering the third composition is performed after administration of the first composition.

37. The method of claim 25, wherein the antigen or immunogen is from a pathogen.

38. The method of claim 37, wherein the pathogen is a virus, fungus, parasite, or bacteria.

39. The method of claim 38, wherein the virus is EBV, HPV, HTLV-1, MCPvV, KSHV, HERV, HCV or HBV.

40. The method of claim 25, wherein the antigen or immunogen is a cancer antigen.

41. The method of claim 25, wherein the induced immune response is a therapeutic or prophylactic treatment for non-small-cell lung cancer, breast cancer, hepatic cancer, brain cancer, stomach cancer, pancreatic cancer, kidney cancer, ovarian cancer, myeloma cancer, acute myelogenous leukemia, chronic myelogenous leukemia, head and neck cancer, colorectal cancer, renal cancer, esophageal cancer, melanoma skin cancer and/or prostate cancer patients.

42. The method of claim 25, wherein the induced immune response is a therapeutic or prophylactic treatment for subjects with a chronic infection or risk of exposure to pathogens that cause chronic infections, wherein the pathogens are selected from human immunodeficiency virus (HIV), hepatitis B virus, hepatitis D viruses, herpesviruses, Epstein-Barr virus, cytomegalovirus, human papilloma virus, or human T-lymphotropic virus type III.

43. The method of claim 26, wherein a route of administration of the first and/or second composition is selected from parenteral, subcutaneous, oral, epidermal, intradermal, intramuscular, intraarterial, intraperitoneal, intravenous, nasally, intratracheally, intestinally, sublingually, rectally or vaginally.

44. The method of claim 25, wherein the non-replicating viral vector is an adenovirus vector, an alphavirus vector, a herpesvirus vector, a measles virus vector, a poxviruses vector, or a vesicular stomatitis virus vector.

45. The method of claim 44, wherein the non-replicating viral vector is an E1 and/or E3 deleted adenovirus vector.

46. The method of claim 25, wherein the fluorocarbon portion of the fluorocarbon-linked peptide is a fluorocarbon chain from 3 to 30 carbon atoms.

47. The method of claim 46, wherein one or more fluorine moieties of the fluorocarbon chain is substituted with chlorine, bromine, iodine, hydrogen or a methyl group.

48. The method of claim 25, wherein the fluorocarbon-linked peptide is of structure CₘFₙ—C_{y}Hₓ-(Sp)-R, wherein m=3 to 30, n<=2m+1, y=0 to 15, x<=2y, (m+y)=3-30, Sp is an optional chemical spacer moiety and R is the peptide.

49. The method of claim 25, wherein the fluorocarbon linked peptide is according to structure 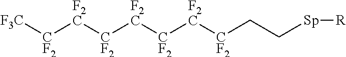
where Sp is an optional chemical spacer moiety and R is the peptide.

50. The method of claim 25, wherein the fluorocarbon linked peptide of the second composition comprises at least one MHC class II binding epitope and at least one MHC class I binding epitope.

51. A method of inducing an immune response in a subject in need thereof, comprising:
a) administering a first composition comprising a non-replicating viral vector encoding an antigen or immunogen, or fragment thereof, containing one or more CD8+ T cell epitopes; or,
b) administering a second composition comprising micelles containing fluorocarbon-linked peptides, wherein each peptide linked to the fluorocarbon is:
i) 15 to 75 amino acid residues long;
ii) from an antigen or immunogen; and,
iii) contains one or more CD8+ T cell epitopes; and,
c) administering separately a third composition comprising an immune modulator selected from an anti-immunosuppressor or an immunoactivator,
whereby an antigen specific CD8+ T cells response is induced.

52. The method of claim 51, wherein the subject is mammal, bird, reptile, amphibian, or fish.

53. The method of claim 51, wherein the subject is a human, a cow, a dog, a cat, a goat, a sheep, a pig, a horse, a turkey, duck or chicken.

54. The method of claim 51, wherein the anti-immunosuppressor targets PD1, PDL1, PDL2, CD28, CD80, CD86, CTLA4, B7RP1, ICOS, B7RPI, B7-H3, B7-H4, BTLA, HVEM, KIR, TCR, LAG3, CD 137, CD137L, OX40, OX40L, CD27, CD70, CD40, CD40L, TIM3, GAL1, GAL3, GALS, ADORA, CD276, VTCN1, IDOL KIR3DL1, HAVCR2, VISTA, CD244, ADAM17, COX2, PGE-2, iNOS2, PDE5, c-kit, ARG1, PI3K, CSF-1R, Caspase-8, CCL2, RON, ROS, or S100A8/A9.

55. The method of claim 51, wherein the anti-immunosuppressor targets PD1 or PDL1.

56. The method of claim 51, wherein the anti-immunosuppressor is an anti-PD1 or anti-PDL1 antibody.

57. The method of claim 51, wherein the immunoactivator targets Toll-like receptor (TLR) 3, TLR4, TLR5, TLR7, TLR8, TLR9, NOD1, NOD2, STING, cGAS, IFR3, IL-2 receptor, IL12 receptor or IFN-alpha receptor.

58. The method of claim 51, wherein the step of administering the third composition is performed after administration of the first or second composition.

59. The method of claim 51, wherein the antigen or immunogen is from a pathogen.

60. The method of claim 59, wherein the pathogen is a virus, fungus, parasite, or bacteria.

61. The method of claim 60, wherein the virus is EBV, HPV, HTLV-1, MCPvV, KSHV, HERV, HCV, or HBV.

62. The method of claim 51, wherein the antigen or immunogen is a cancer antigen.

63. The method of claim 51, wherein the induced immune response is a therapeutic or prophylactic treatment for non-small-cell lung cancer, breast cancer, hepatic cancer, brain cancer, stomach cancer, pancreatic cancer, kidney cancer, ovarian cancer, myeloma cancer, acute myelogenous leukemia, chronic myelogenous leukemia, head and neck cancer, colorectal cancer, renal cancer, esophageal cancer, melanoma skin cancer and/or prostate cancer patients.

64. The method of claim 51, wherein the induced immune response is a therapeutic or prophylactic treatment for subjects with a chronic infection or risk of exposure to pathogens that cause chronic infections, wherein the pathogens are selected from human immunodeficiency virus (HIV), hepatitis B virus, hepatitis D viruses, herpesviruses, Epstein-Barr virus, cytomegalovirus, human papilloma virus, or human T-lymphotropic virus type III.

65. The method of claim 51, wherein a route of administration of the first or second composition and third composition is independently selected from parenteral, subcutaneous, oral, epidermal, intradermal, intramuscular, intraarterial, intraperitoneal, intravenous, nasally, intratracheally, intestinally, sublingually, rectally or vaginally.

66. The method of claim 51, wherein the non-replicating viral vector is an adenovirus vector, an alphavirus vector, a herpesvirus vector, a measles virus vector, a poxviruses vector, or a vesicular stomatitis virus vector.

67. The method of claim 66, wherein the non-replicating viral vector is an E1 and/or E3 deleted adenovirus vector.

68. The method of claim 51, wherein the fluorocarbon portion of the fluorocarbon-linked peptide is a fluorocarbon chain from 3 to 30 carbon atoms.

69. The method of claim 68, wherein one or more fluorine moieties of the fluorocarbon chain is substituted with chlorine, bromine, iodine, hydrogen or a methyl group.

70. The method of claim 51, wherein the fluorocarbon-linked peptide is of structure CₘFₙ—C_{y}Hₓ-(Sp)-R, wherein m=3 to 30, n<=2m+1, y=0 to 15, x<=2y, (m+y)=3-30, Sp is an optional chemical spacer moiety and R is the peptide.

71. The method of claim 51, wherein the fluorocarbon linked peptide is according to structure 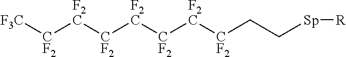
where Sp is an optional chemical spacer moiety and R is the peptide.

72. The method of claim 51, wherein the fluorocarbon linked peptide of the second composition comprises at least one MHC class II binding epitope and at least one MHC class I binding epitope.
